(19) United States
(12) Patent Application Publication
(10) Pub. No.: US 2024/0277913 A1
(43) Pub. Date: Aug. 22, 2024

(54) EXTRACORPOREAL DISINFECTION SYSTEM

(71) Applicant: Radiatric Inc., Washougal, WA (US)

(72) Inventors: Jorel Lalicki, Troy, NY (US); Stephen J. Petti, Troy, NY (US); Ron Tribble, Washougal, WA (US); Olivia Jackson, Avon, CT (US)

(21) Appl. No.: 18/562,052

(22) PCT Filed: May 9, 2022
(86) PCT No.: PCT/US22/28314

§ 371 Date: Nov. 17, 2023

Related U.S. Application Data
(60) Provisional application No. 63/189,748, filed on May 18, 2021.

(51) Int. Cl. A61M 1/36; A61L 2/00

(52) U.S. Cl. CPC A61M 1/3681; A61L 2/0052; A61L 2202/10; A61L 2202/22; A61M 2202/0021; A61M 2202/0413; A61M 2205/0238; A61M 2205/10; A61M 2205/18; A61M 2205/3327; A61M 2205/3334; A61M 2205/3368; A61M 2205/3606; A61M 2205/362; A61M 2205/3633; A61M 2205/366; A61M 2205/702; A61M 2205/7545; A61M 2209/02

(57) ABSTRACT

An extracorporeal blood disinfection system (10) includes an input tube (12) forming a flowpath (22) for infected blood from a mammalian patient (11); a disinfection unit (14) including a microbicidal light emitting device (24) emitting a plurality of light emissions, each light emission having a wavelength in a range between about 380 to about 800 nm; a treatment flowpath (22) in communication with the input tube (12) that is substantially transparent to emitted light of the microbicidal light emitting device (24) for receiving at least a portion of the infected blood flow therethrough, wherein the microbicidal light emitting device (24) effectuates a dose of light emissions to the infected blood in the treatment flowpath (22) to disinfect the blood; and an output tube (16) fluidly and physically connected wherein material can flow within the treatment flowpath forming a flowpath the disinfected blood flow from the disinfection unit to the patient.

### BACKGROUND

Extracorporeal fluid treatment typically involves the removal of fluid from a patient, treatment of the external/removed fluid, and return of the treated fluid into the patient in a continuous circuit or path. Blood is one body fluid for which conventional extracorporeal techniques have been developed. Using such techniques, blood is typically treated to extract materials from the blood and/or add materials to augment the blood prior to return of the treated blood to the patient. In some techniques, blood is removed from the patient in a continuous flow and introduced into a chamber containing a filtration unit where the blood flows past a semipermeable membrane. The semipermeable membrane allows only select material in the blood to pass through the membrane for removal from the blood (or removal of the material that does not pass therethrough) and/or allows material to pass through the semipermeable membrane and into the blood. After passage of material to and/or from the blood, the treated blood is discharged from the filtration unit for return to the patient. If material has been removed from the blood, the removed material is separately discharged from the filtration unit.

One ailment that includes pathogens within a mammal's blood that is not satisfactorily treated by conventional extracorporeal techniques is sepsis. Acute sepsis is a metastatic infection that arises when infectious microbes in the circulatory system overwhelm the immune system and the pathogenic microorganisms can no longer be removed from circulating blood (by the body) faster than they are proliferating. Sepsis thus originates as an isolated infection of pathogenic microorganisms that become mobile in the circulatory system. When bacteria or viruses are present in the bloodstream, the condition is known as bacteremia or viremia. Pathogenic fungi can also be present in the bloodstream. Sepsis is a constellation of symptoms secondary to the infection that manifest as disruptions in heart rate, respiratory rate and/or body temperature. Bacteremia is rarely associated with any signs or symptoms, and most pathogenic microorganisms are readily removed from circulation by the humoral immune system.

When pathogenic microbes begin to reproduce in the circulatory system and the body is unable to remove them at an adequate rate, septicemia develops, and a systemic inflammatory response is initiated. This syndrome, SIRS, is one of the primary disease patterns in sepsis. If acute sepsis worsens to the point of end-organ dysfunction (such as renal failure, liver dysfunction, altered mental status or heart damage), the condition is referred to as “severe sepsis.” Once severe sepsis worsens to the point where blood pressure can no longer be maintained by the body with intravenous fluid intervention alone, then the criteria for “septic shock” are met. Precipitating infections which may lead to septic shock, if severe enough, include appendicitis, pneumonia, bacteremia, diverticulitis, pyelonephritis, meningitis, pancreatitis, and necrotizing fasciitis, for example.

It has been reported that acute sepsis kills approximately 120,000-200,000 people annually in the United States. Statistics have shown that sepsis is the second leading cause of death in non-coronary ICU patients. Overall, sepsis is the tenth most common cause of death in the United States and accounts for as much as 25% of ICU bed utilization, according to some reports. In fact, it has been said that the medical specialty practice of Critical Care Management was specifically developed as a result of septic mortality rates. According to some reports, overall sepsis mortality is in the range of 40-44%, with mortality rates rising to between 70-90% when sepsis proceeds to severe septicemia or septic shock (i.e., proceeds to organ dysfunction, hypotension, or hypo-perfusion, or to hypotension despite adequate fluid resuscitation, respectively).

Septic shock, the most severe manifestation of sepsis, is a clinical syndrome that results, at least in part, from an activated systemic host inflammatory response to infection leading to cardiovascular collapse. Septic shock has become increasingly common in the North American population, potentially because of an increasing population of at risk elderly individuals (many of whom have chronic debilitating disease) and individuals with impaired immunity due to diseases (such as cancer and AIDS). According to some reports, about 750,000 cases of sepsis occur each year in the United States.

Common origins of infections that develop into sepsis include urinary tract infections, pneumonia, cellulitis, wounds and abscesses, sinusitis, meningitis, and surgical procedures to an infected area or the abdomen. The most common causative organisms associated with sepsis can include Gram-positive bacteria (e.g., Staphylococcus aureus, coagulase-negative Staphylococcus, Streptococcus pyogenes, Streptococcus pneumoniae, and enterococci), Gram-negative bacteria (e.g., Proteus, Serratia, Pseudomonas aeruginosa, Neisseria meningitudis, and Escherichia coli Klebsiella pneumoniae), anaerobic organisms and fungi (e.g., Candida albicans).

The progression from sepsis to septic shock follows the significant increase in serum levels of TNF-a, IFN-a, IL-1B, IL-8, and IL-6. Shock is a condition defined by inadequate tissue perfusion, such as resulting from vasodilation due to increased cytokine levels and intravascular fluid shifting. Increase in TNF-a levels is believed to be responsible for the onset of disseminated intravascular coagulation (DIC). These conditions, together with renal and liver failure, can cause cardiac collapse and respiratory failure (ARDS).

The signs and symptoms of sepsis vary according to the associated underlying disease/infection process(es). However, most symptomology is universal. Sepsis is typically preceded by a period of altered mental status (e.g., for approximately 24 hours) before other signs develop. Fatigue, malaise, myalgia, nausea, and vomiting are common early signs. Fever typically initially develops, but often declines to hypothermia in late stages. Elevated heart rate and respiratory rate typically develop as blood pressure becomes erratic. Blood pressure often eventually declines dramatically as plasma shifts and vasodilation worsens. Impaired renal function is typically evident with decreased urinary output, as is liver failure with jaundice.

No universally effective, curative treatments currently exist for sepsis, severe septicemia, and septic shock. Some supportive measures, such as antibiotics, vasopressor agents and various endogenous strategies associated with attempts at blood “purification,” are associated with some degree of lower mortality in patients with sepsis (primarily with treatments utilizing polymyxin B). For example, antibiotics are often inadequate to combat the overwhelming invasion of bacteria or other infectious microorganisms associated with the more severe manifestations of sepsis. In addition, antibiotic resistant strains of bacteria are prevalent, limiting the effectiveness of antibiotic treatment. Vasopressor agents may support hypotension associated with cardiovascular collapse, but fail to treat the infectious microorganisms. Current attempts at blood purification, which include hemoperfusion, plasma exchange, and hemofiltration with hemoperfusion, inadequately remove the infectious microorganisms associated with sepsis, severe septicemia, and septic shock. As such, despite all current treatment approaches, mortality from all manifestations and degrees of severity of sepsis remains unacceptably high.

While certain aspects of conventional technologies have been discussed to facilitate disclosure of Applicant's disclosure, the Applicant in no way disclaims these technical aspects, and it is contemplated that their disclosure may encompass one or more conventional technical aspects. In this specification, where a document, act or item of knowledge is referred to or discussed, this reference or discussion is not an admission that the document, act or item of knowledge or any combination thereof was, at the priority date, publicly available, known to the public, part of common general knowledge, or otherwise constitutes prior art under the applicable statutory provisions: or is known to be relevant to an attempt to solve any problem with which this specification is concerned.

### BRIEF DESCRIPTION

The present disclosure describes a system and method for disinfecting blood utilizing multiple wavelengths of light. In various embodiments, the systems and methods are particularly advantageous for treatment and/or prevention of sepsis. The present disclosure may address one or more of the problems and deficiencies of the art discussed above. However, it is contemplated that the disclosure may prove useful in addressing other problems and deficiencies in a number of technical areas. Therefore, the claims should not necessarily be construed as limited to addressing any of the particular problems or deficiencies discussed herein.

In some embodiments, the disinfection systems and methods of the present disclosure comprise an extracorporeal device designed to address pathogenic microbial infection associated with sepsis (including severe sepsis and septic shock) by externally reducing or eliminating the presence of the infectious microorganisms associated with the condition(s) in a mammal's blood. The systems and methods also provide an effective and unique stand-alone or adjunctive therapeutic intervention to standard measures used in the treatment of sepsis (including severe sepsis and septic shock), such as compared to anti-infective drug therapy, to combat the overwhelming infectious process characteristic of the condition wherein infecting microbial organisms can no longer be removed from circulating blood faster than they are proliferating.

In some embodiments, the disinfection systems and methods of the present disclosure comprise a blood pump with a built in or attached microbicidal lighting device intended to expose a blood flow of mammalian blood at sufficient intensity and duration that is effective in reducing or eliminating or slowing the proliferation of a broad range of infectious sources, such as infectious sources known to be associated with sepsis (e.g., severe sepsis and/or septic shock).

In some embodiments, the device may include an attached or incorporated filter designed to remove one or more unwanted by-product of disinfection or one or more toxins released by an infectious microorganisms from the blood before returning the blood to the patient's body, and/or an attached or incorporated infusion component to that restores or supplements one or more component into the blood that is/may be affected by the microbicidal process and/or by the infectious agents/microorganisms.

In some embodiments, the device may include an optical element associated with the lighting device configured to scatter, direct, or combine light from one or more irradiation sources resulting in uniform intensity of the light incident on the blood. In some embodiments, the device may include one or more reflectors configured to reflect stray light back into a treatment pathway of the blood and/or to improve uniformity of irradiation. In some embodiments, the device may include a cooling device (such as a heatsink, waterblock, peltier cooling system, heatpipe, or phase change material element) associated with the lighting device and configured to dissipate heat from the lighting device to maintain the temperature of the lighting device below a predefined temperature such that the lighting device is prevented from heating the treated blood above a predefined temperature, or to maintain a preferred operating temperature for the light engine, such as to maximize efficiency, prevent damage, or maintain a characterized light output.

FIG. 3 depicts the absorption of porphyrin molecules across a range of wavelengths. In some embodiments, the disinfection systems and methods of the present disclosure include at least one microbicidal lighting device or lighting mechanism that emits a plurality of light emissions having different wavelengths in at least two of the porphyrin absorption peaks B/Soret and Q bands e.g., the 380-425 nm wavelength (violet) range and four peaks within the 500 to 700 nm range, as indicated in FIG. 3. These light spectrums can disinfect and/or sterilize mammalian blood to treat infections that typically cause or result in sepsis (i.e., kill pathogenic microorganisms and/or prevent such microorganisms from reproducing that cause, or tend to cause, sepsis). These light spectrums are otherwise substantially safe to mammalian blood. For example, these light spectrums are absorbed by single cell organisms (e.g., non-mammalian cells) (specifically, by the forms of porphyrin found within organisms thereof, for example) and cause the population of a triplet state. The triplet state results in the creation of a ‘reactive oxygen species’ (ROS)—a singlet oxygen molecule, radical species, superoxide anions, free hydroxyl radicals, and/or hydrogen peroxide which can serve to inactivate the organism by reacting with susceptible cellular biomolecules such as lipids, amino acids, and nucleic acid heterocyclic bases.

FIG. 5 is a graph of experimental data showing a reduction in colony forming units (CFU) in transparent broth over time with exposure to selected wavelengths. The experimental data shows a significant reduction in CFU with all wavelengths, however, the efficacy of the 405 nm treatment is shown to be substantially higher than the efficacy of 630 and 663 nm, which is shown to be substantially higher than 596 nm. These measured efficacies correspond to the expected absorption peaks as shown in FIG. 3.

In some embodiments, the disinfection systems and methods of the present disclosure may include a blood pump with a built in or attached microbicidal lighting device intended to expose blood flow at sufficient intensity and duration (i.e., total dose) that is effective in reducing or eliminating or slowing the proliferation of a broad range of infectious sources, such as those associated with sepsis (including severe sepsis and/or septic shock).

In one embodiment, the device may include an attached or built in filter designed to remove any potential toxins released by the microorganisms and/or unwanted by-products of disinfection before returning the patient's blood to their body, and/or an attached or incorporated infusion component configured to restore or supplement one or more blood components of the treated blood that is negatively affected by the treatment proves (e.g., negatively affected by the microbicidal light).

All aspects, examples and features mentioned below can be combined in any technically possible way.

An aspect of the disclosure provides an extracorporeal blood disinfection system, comprising: an input tube forming a flowpath for the flow of infected blood from a mammalian patient: a disinfection unit including a microbicidal light emitting device configured to emit a plurality of light emissions, each light emission having a wavelength within the range of about 380 nm to about 800 nm: a treatment flowpath in communication with the input tube that is substantially transparent to the emitted light of the microbicidal light emitting device for receiving at least a portion of the flow of the infected blood therethrough, wherein the microbicidal light emitting device effectuates a dose of the plurality of light emissions to the infected blood flowing through the treatment flowpath to disinfect the blood; and an output tube in fluid communication with the treatment flowpath forming a flowpath for the flow of the disinfected blood from the disinfection unit back to the patient.

Another aspect of the disclosure includes any of the preceding aspects, and further comprising a pump configured to at least one of: provide the flow of infected blood from the patient through the input tube: pass the blood from the input tube through the treatment flowpath; and provide the flow of disinfected blood to the patient through the output tube.

Another aspect of the disclosure includes any of the preceding aspects, and the disinfection unit further comprises a thermal management device associated with the microbicidal light emitting device and configured to maintain the blood within the treatment pathway above a blood temperature set point.

Another aspect of the disclosure includes any of the preceding aspects, and the thermal management device includes a treatment flowpath coolant flowpath thermally coupled with the treatment flowpath, the treatment flowpath coolant flowpath including one or more channels configured to allow a coolant to flow therethrough.

Another aspect of the disclosure includes any of the preceding aspects, and the treatment flowpath coolant flowpath is optically transparent, thermally coupled with the treatment flowpath, and thermally isolated from the microbicidal light emitting device.

Another aspect of the disclosure includes any of the preceding aspects, and further comprising a blood temperature sensor configured to measure a blood temperature of the blood, wherein the thermal management device controls a temperature of the coolant based on the blood temperature.

Another aspect of the disclosure includes any of the preceding aspects, and further comprising a heat exchanger configured to control the temperature of the coolant based on the blood temperature.

Another aspect of the disclosure includes any of the preceding aspects, and the thermal management device controls a blood flow rate generated by the pump based on the blood temperature.

Another aspect of the disclosure includes any of the preceding aspects, and further comprising a fault detection system configured generate an alarm in response to at least one of: the blood temperature exceeding a blood temperature set point: a temperature of the microbicidal light emitting device being outside of an allowable range; and detection of an air bubble in the treatment flowpath.

Another aspect of the disclosure includes any of the preceding aspects, and the disinfection unit further comprises a thermal management device associated with the microbicidal light emitting device configured to dissipate heat from the microbicidal light emitting device to maintain the temperature of at least a portion thereof below a predefined temperature such that the microbicidal light emitting device is prevented from heating the blood within the treatment pathway above a blood temperature set point.

Another aspect of the disclosure includes any of the preceding aspects, and further comprising an insulating layer between the microbicidal light emitting device and the treatment flowpath.

Another aspect of the disclosure includes any of the preceding aspects, and the insulating layer is optically transparent, and resistant to conductive heat transfer.

Another aspect of the disclosure includes any of the preceding aspects, and the insulating layer includes one of: air, a vacuum, a gas, aerogels, a material configured to trap air pockets, such as but not limited to, glass wool, and a polymeric material.

Another aspect of the disclosure includes any of the preceding aspects, and the insulating layer includes an optical diffuser.

Another aspect of the disclosure includes any of the preceding aspects, and the insulating layer includes a surface that reflects or resists transmission of infrared thermal energy.

Another aspect of the disclosure includes any of the preceding aspects, and the insulating layer includes at least one optical feature to direct the plurality of light emissions.

Another aspect of the disclosure includes any of the preceding aspects, and the dose of light is effective in at least one of: eliminating pathogenic microorganisms from the infected blood; partially reducing the number of the pathogenic microorganisms in the infected blood; and reducing the rate of proliferation of the pathogenic microorganisms in the infected blood.

Another aspect of the disclosure includes any of the preceding aspects, and the pathogenic microorganisms comprise microorganisms associated with at least one of sepsis, severe sepsis, and septic shock.

Another aspect of the disclosure includes any of the preceding aspects, and the pathogenic microorganisms comprise at least one of bacteria, fungi, yeast, and a combination thereof.

Another aspect of the disclosure includes any of the preceding aspects, and the pathogenic microorganisms comprise at least one of gram positive bacteria, gram negative bacteria, bacterial endospores, yeast, filamentous fungi, and a combination thereof.

Another aspect of the disclosure includes any of the preceding aspects, and the pathogenic microorganisms comprise at least one of Staphylococcus aureus, Clostridium perfringens, Clostridium difficile, Enterococcus faecalis, Staphylococcus epidermidis, Staphylococcus hyicus, Streptococcus pyogenes, Listeria monocytogenes, Bacillus cereus, Mycobacterium terrae, Acinetobacter baumannii, Pseudomonas aeruginosa, Klebsiella pneumoniae, Proteus vulgaris, Escherichia coli, Salmonella enteritidis, Shigella sonnei, Serratia spp, Bacillus cereus, Clostridium difficile, Aspergillus niger, Candida albicans, Saccharomyces cerevisiae and a combination thereof.

Another aspect of the disclosure includes any of the preceding aspects, and the plurality of light emissions includes light within at least two ranges of: about 380 nm to about 420 nm; about 400 nm to about 415 nm: about 405 nm; about 500 nm to about 700 nm; about 500 nm to about 520 nm: about 530 nm to about 555 nm, about 565 nm to about 590 nm and about 615 nm to about 645 nm.

Another aspect of the disclosure includes any of the preceding aspects, and the mammalian patient is a human patient.

Two or more aspects described in this disclosure, including those described in this summary section, may be combined to form implementations not specifically described herein.

The details of one or more implementations are set forth in the accompanying drawings and the description below. Other features, objects and advantages will be apparent from the description and drawings, and from the claims.

### BRIEF DESCRIPTION OF THE DRAWINGS

The present disclosure will hereinafter be described in conjunction with the following drawing figures, which are not necessarily drawn to scale for ease of understanding, wherein the same reference numerals retain their designation and meaning for the same or like elements throughout the various drawings, and wherein:

FIG. 1 is a diagram illustrating an example extracorporeal blood disinfection system of the present disclosure;

FIG. 2 is a perspective view of the extracorporeal blood disinfection system of FIG. 1;

FIG. 3 is a graph showing experimental data of absorption of light into porphyrin molecules at various wavelengths;

FIG. 4 is a graph showing experimental data of wavelength dependent transmission of light through various depths of human whole blood;

FIG. 5 is a graph showing experimental data of reduction of bacterial colonies over time with exposure to various wavelengths of light;

FIG. 6 is top view of a microbicidal light emitting device and treatment pathway of the extracorporeal blood disinfection system of FIGS. 1 and 2;

FIG. 7 is a side view of the light emitting device and treatment pathway of FIG. 6;

FIG. 8 is top view of a microbicidal light emitting device and treatment pathway of another extracorporeal blood disinfection system of the present disclosure;

FIG. 9 is a side view of the light emitting device and treatment pathway of FIG. 8;

FIG. 10 is top view of a treatment pathway of another extracorporeal blood disinfection system of the present disclosure;

FIG. 11 is a side view of a microbicidal light emitting device and the light emitting device of FIG. 10;

FIG. 12 is a perspective view of a microbicidal light emitting device and treatment pathway of another extracorporeal blood disinfection system of the present disclosure;

FIG. 13 is an elevational perspective view of a microbicidal light emitting device and treatment pathway of another extracorporeal blood disinfection system;

FIG. 14 is top view of the light extracorporeal blood disinfection system of FIG. 13;

FIG. 15 is top view of a portion of the extracorporeal blood disinfection system of FIG. 13;

FIG. 16 is a side view of a light emitting device of the extracorporeal blood disinfection system of FIG. 13;

FIG. 17 is a side view of a light emitting device of the extracorporeal blood disinfection system of FIG. 13 including control system aspects;

FIG. 18 is a diagram of a light emitting device utilizing laser sources and a combining element;

FIG. 19 is a side partial cross-sectional view of a microbicidal light emitting device and treatment pathway of another extracorporeal blood disinfection system;

FIG. 20 is a side view of a portion of the treatment pathway and emitter shaft of the light emitting device of FIG. 19;

FIG. 21 is an elevation, perspective view of a microbicidal light emitting device and treatment pathway of another extracorporeal blood disinfection system;

FIG. 22 is a cross-sectional view of the light emitting device and treatment pathway of FIG. 21;

FIG. 23 is a cross-sectional view of the light emitting device and treatment pathway including a blood temperature regulation element; and

FIG. 24 is a schematic showing temperature regulation components of the extracorporeal blood disinfection system.

It is noted that the drawings of the disclosure are not necessarily to scale. The drawings are intended to depict only typical aspects of the disclosure and therefore should not be considered as limiting the scope of the disclosure. In the drawings, like numbering represents like elements between the drawings.

### DETAILED DESCRIPTION

Aspects of the present disclosure and certain features, advantages, and details thereof, are explained more fully below with reference to the non-limiting embodiments illustrated in the accompanying drawings. Descriptions of well-known materials, fabrication tools, processing techniques, etc., are omitted so as to not unnecessarily obscure the details of the disclosure. It should be understood, however, that the detailed description and the specific example(s), while indicating embodiments of the present disclosure, are given by way of illustration only, and are not by way of limitation. Various substitutions, modifications, additions and/or arrangements within the spirit and/or scope of the underlying inventive concepts will be apparent to those skilled in the art from this disclosure.

Approximating language, as used herein throughout disclosure, may be applied to modify any quantitative representation that could permissibly vary without resulting in a change in the basic function to which it is related. Accordingly, a value modified by a term or terms, such as “about” or “substantially,” is not limited to the precise value specified. For example, these terms can refer to less than or equal to +5%, such as less than or equal to +2%, such as less than or equal to #1%, such as less than or equal to +0.5%, such as less than or equal to +0.2%, such as less than or equal to +0.1%, such as less than or equal to +0.05%. In some instances, the approximating language may correspond to the precision of an instrument for measuring the value.

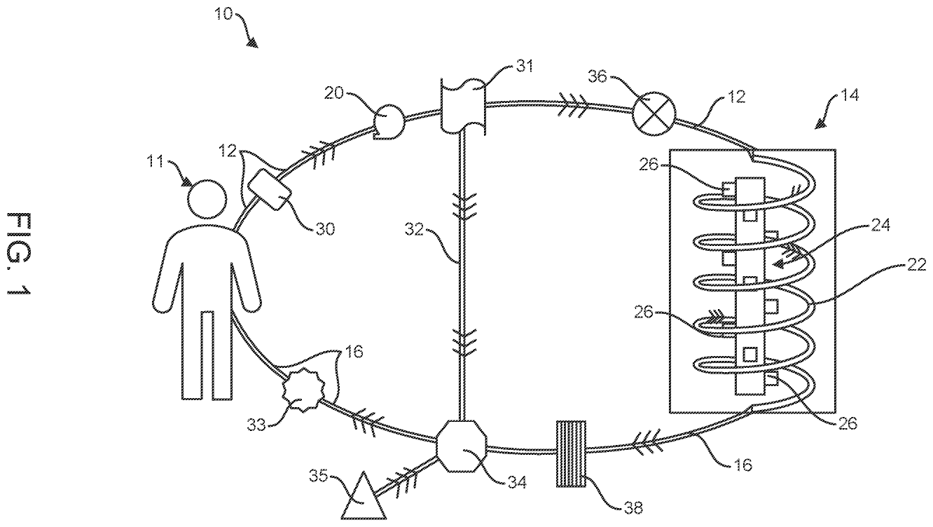

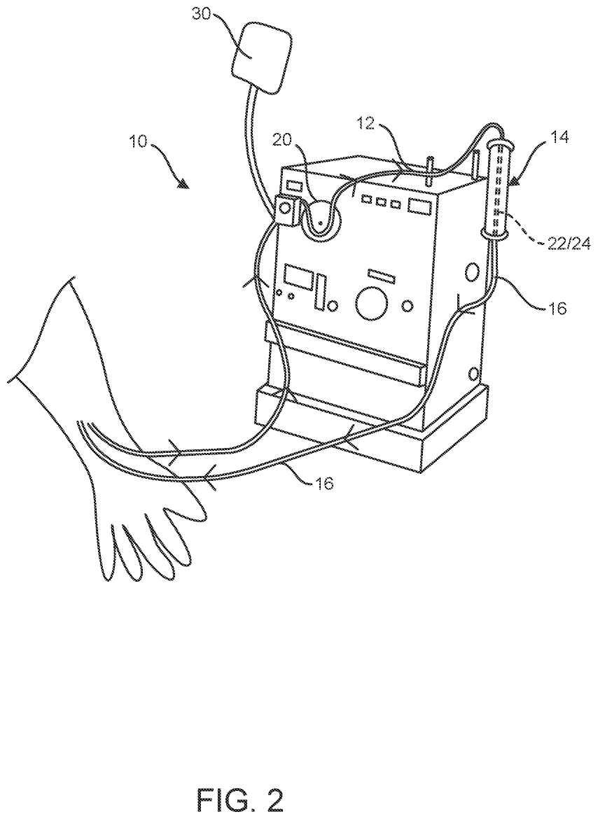

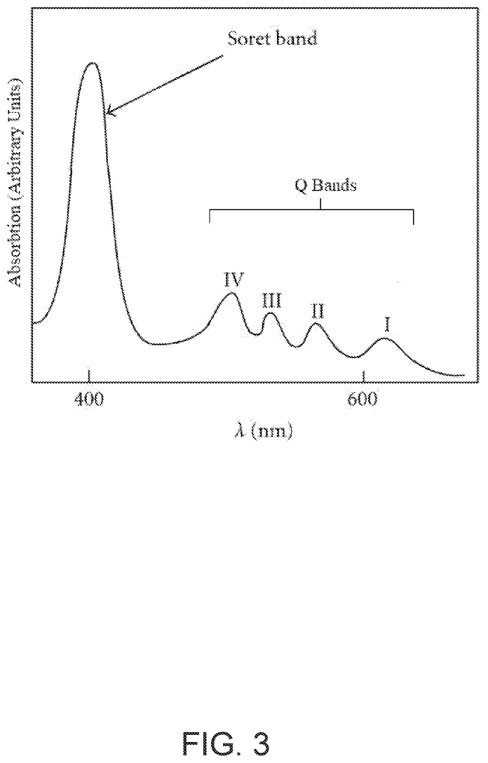

As shown in FIGS. 1 and 2, in some illustrative embodiments a disinfection system and related method 10 of the present disclosure may include an extracorporeal disinfection device 14 comprising a microbicidal light emitting device 24 configured to emit a plurality of light emissions having different wavelengths, e.g., of visible light within the range of about 380 nm to about 425 nm and about 500 nm to about 700 nm. The plurality of light emissions may be selectively absorbed by single cell organisms (e.g., non-mammalian cells) and not by mammalian multi-cell organisms. They system also includes a treatment flowpath 22 that is substantially transparent to the emitted plurality of light emissions having different wavelengths of the microbicidal light emitting device 24 for receiving at least a portion of a flow of infected blood from a patient 11. These light spectrums can be selectively absorbed by single cell organisms (e.g., non-mammalian cells) (such as by the porphyrin thereof, for example). These light spectrums may also be absorbed by specific cells or components of cells in mammalian multi-cell organisms, where the applicant submits that differences in porphyrin structure (vs. single cell organisms), repair mechanisms (including antioxidant compounds), and decreased amount of porphyrin molecules result in no substantial deactivation or damage to the mammalian cells. The forms of porphyrin found within mammalian cells are generally structurally distinct from those forms found in single cell organisms (for example, include heme, a metalloporphyrin, which uniquely contains an iron ion) and do not generally result in deactivating the mammalian cells when exposed to the light bands noted above. Additionally, the structural differences and unique cellular repair mechanisms in mammalian cells (as compared to single cell organisms for example) results in minimal damage to mammalian cells incidentally exposed to disinfecting wavelengths, whereas single cell organisms receiving a similar dose can be significantly deactivated. As used within this disclosure, the term porphyrin (and its linguistic variants) specifically refers to the porphyrin molecules found within single cell organisms, and especially those porphyrin molecules that can be induced to effect cellular deactivation through activation with certain wavelengths of light as described herein.

The extracorporeal disinfection device 14 may expose a blood flow to such light wavelengths at sufficient intensity and duration that the light is effective in reducing or eliminating or slowing the proliferation of a broad range of infectious sources, such as infectious sources known to be associated with sepsis (e.g., severe sepsis) and/or septic shock. In some embodiments, the dose of light emitted from the at least one microbicidal lighting device of the systems and methods may be configured to destroy, eradicate and/or reduce the number of harmful pathogenic microorganisms, or reduce of the rate of proliferation thereof, within the blood or blood portion/derivative that are associated with sepsis (including severe sepsis and septic shock). For example, in some embodiments, the dose of light emitted from the at least one microbicidal lighting device may be configured to destroy, eradicate and/or reduce the number of at least the pathogenic microorganisms listed below in TABLE 1, or reduce of the rate of proliferation thereof, within the treated blood.

TABLE 1

| | | | Yeast and |
|---|---|---|---|
| Gram-Negative | Gram-Positive | Bacterial | Filamentous |
| Bacteria | Bacteria | Endospores | Fungi |
| Acinetobacter | Staphylococcus | Bacillus | Aspergillus |
| baumannii | aureus (incl. MRSA) | cereus | niger |
| Pseudomonas | Clostridium | Clostridium | Candida |
| aeruginosa | perfringens | difficile | albicans |
| Klebsiella | Clostridium difficile | | Saccharomyces |
| pneumoniae | | | cerevisiae |
| Proteus vulgaris | Enterococcus faecalis | | |
| Escherichia coli | Staphylococcus | | |
| | epidermidis (CONS) | | |
| Salmonella | Staphylococcus hyicus | | |
| enteritidis | (CONS) | | |
| Shigella sonnei | Staphylococcus | | |
| | pyogenes | | |
| Serratia spp | Listeria | | |
| | monocytogenes | | |
| | Bacillus cereus | | |
| | Mycobacterium | | |
| | terrae | | |

Continuing with FIG. 1, patient 11 may be a mammalian patient. For example, patient 11 may be a human. As another example, patient 11 may be a non-human mammal, such an endangered species being rehabilitated, or any other non-human mammalian animal.

System 10 and/or the extracorporeal disinfection device 14 itself is configured such that the microbicidal light emitting device 24 externally (with respect to the patient 11) effectuates a dose of the emitted light to the infected blood flowing through the treatment flowpath 22 that reduces or eliminates the presence of infectious microorganisms in the blood that are commonly associated with sepsis, severe sepsis and/or septic shock to address a pathogenic microbial infection of the patient 11 that is leading to, or could/would lead to, sepsis, severe sepsis and/or septic. The system 10 and treatment method associated therewith may thereby provide a stand-alone or adjunctive therapeutic intervention to standard measures used in the treatment of sepsis, severe sepsis and/or septic shock to combat the overwhelming infectious process characteristic of the conditions where infecting microbial organisms can no longer be removed from circulating blood by the body's natural processes faster than they are proliferating. For example, the system 10 and treatment method associated therewith may be utilized in conjunction with an anti-infective drug therapy.

In some embodiments, the microbicidal lighting device 24 of the extracorporeal disinfection device 14 of the disinfection system 10 is configured to emit light in the 380-420 nm (violet) range. In some such embodiments, the light spectrum emitted from the microbicidal lighting device 24 may thereby be configured as bactericidal, but yet safe for processing the blood of a patient 11 without critically damaging the blood when infecting microbial organisms can no longer be removed from circulating blood by the patient 11 faster than they are proliferating. As noted above, the light emitted from the microbicidal lighting device 24 is of wavelengths that may be selectively absorbed by single cell organisms (e.g., non-mammalian cells) (such as by the porphyrin thereof, for example), and may not be absorbed by mammalian multi-cell organisms (as they are void, or at least substantially void, of porphyrin, for example). In some embodiments, system 10 is configured to effectuate a dose of the light emitted from the microbicidal light emitting device 24 to the infected blood of the patient 11 flowing through the treatment flowpath 22 that destroys, eradicates and/or reduces the number of harmful pathogenic microorganisms, or reduces the rate of proliferation thereof, within the blood or blood products/derivatives, such as those that cause sepsis (e.g., tend to cause, known to cause or may cause sepsis). For example, in some embodiments, the system 10 is configured to effectuate a dose of the light emitted from the microbicidal light emitting device 24 to the infected blood of the patient 11 flowing through the treatment flowpath 22 that destroys, eradicates and/or reduces, or reduces the rate of the proliferation of, infectious microorganisms in the blood that (can or likely) cause, for example, UTI, pneumonia, cellulitis, wounds and abscesses, sinusitis and/or meningitis. In some embodiments, the system 10 is configured to effectuate a dose of the light emitted from the microbicidal light emitting device 24 to the infected blood of the patient 11 flowing through the treatment flowpath 22 that destroys, eradicates and/or reduces, or reduces the rate of the proliferation of, for example, gram-positive bacteria (e.g., Staphylococcus aureus, coagulase-negative Staphylococcus, Streptococcus pyogenes, Streptococcus pneumoniae, and enterococci), gram-negative bacteria (e.g., Proteus, Serratia, Pseudomonas aeruginosa, Neisseria meningitidis, Escherichia coli Klebsiella pneumoniae), anaerobic organisms, fungi (e.g., Candida albicans) and/or combinations thereof.

The system 10 may thereby be configured to effectuate a dose of the light emitted from the microbicidal light emitting device 24 to the infected blood of the patient 11 flowing through the treatment flowpath 22 effective in at least one of: selectively eliminating pathogenic microorganisms from the infected blood: selectively partially reducing the number of the pathogenic microorganisms in the infected blood; and selectively reducing the rate of proliferation of the pathogenic microorganisms in the infected blood. The pathogenic microorganisms may comprise microorganisms associated with at least one of sepsis, severe sepsis, and septic shock to treat and/or prevent at least one of sepsis, severe sepsis, and septic shock. For example, in some embodiments, the pathogenic microorganisms may comprise at least one of bacteria, fungi, yeast and a combination thereof. In some such embodiments, the pathogenic microorganisms may comprise at least one of gram positive bacteria, gram negative bacteria, bacterial endospores, yeast, filamentous fungi, and a combination thereof. In some such embodiments, the pathogenic microorganisms may comprise at least one of Staphylococcus aureus, Clostridium perfringens, Clostridium difficile, Enterococcus faecalis, Staphylococcus epidermidis, Staphylococcus hyicus, Streptococcus pyogenes, Listeria monocytogenes, Bacillus cereus, Mycobacterium terrae, Acinetobacter baumannii, Pseudomonas aeruginosa, Klebsiella pneumoniae, Proteus vulgaris, Escherichia coli, Salmonella enteritidis, Shigella sonnei, Serratia spp. Bacillus cereus, Clostridium difficile, Aspergillus niger, Candida albicans, Saccharomyces cerevisiae and a combination thereof.

In some embodiments, the microbicidal light emitting device 24 of the extracorporeal disinfection device 14 is configured to emit light within the range of about 380 nm to about 420) nm. In some embodiments, the microbicidal light emitting device 24 of the extracorporeal disinfection device 14 is configured to emit light within the range of about 400 nm to about 415 nm. In some such embodiments, the microbicidal light emitting device 24 of the extracorporeal disinfection device 14 is configured to emit light of about 405 nm. In some embodiments, the microbicidal light emitting device 24 of the extracorporeal disinfection device 14 is configured to emit light within the range about 500 nm to about 700 nm.

In some embodiments, the microbicidal light emitting device 24 of the extracorporeal disinfection device 14 is configured to emit light within the range of at least one of about 500 nm to about 520 nm, about 530) nm to about 555 nm, about 565 nm to about 590) nm and about 615 nm to about 645 nm. In some such embodiments, the microbicidal light emitting device 24 of the extracorporeal disinfection device 14 is configured to emit light within the range of about 500 nm to about 520 nm, within the range of about 530 nm to about 555 nm, within the range of about 565 nm to about 590 nm and/or within the range of about 615 nm to about 645 nm. The light emitted from the microbicidal lighting device 24 may thereby be of one or more wavelengths that may be selectively absorbed by single cell organisms (e.g., non-mammalian cells) (such as by the porphyrin thereof, for example), as opposed to being absorbed by mammalian multi-cell organisms (as they are void, or at least substantially void, of porphyrin, for example).

As shown in FIGS. 1 and 2, the disinfection system 10 and related treatment methods may function similar to a dialysis loop procedure where blood is removed from the patient 11 via an input passageway, tube, or flow 12 which has been placed in, or otherwise fluidly coupled to, a blood vessel (e.g., a vein or artery) of the patient 11. The input tube 12 may be threaded through/to an extracorporeal blood pumping device 20 and extend in relatively close physical proximity to the extracorporeal disinfection device 14 such that at least some of the emitted light thereof is incident on the blood.

The input tube 12 and the output tube 16 may each comprise any tube, hose or other mechanism that forms at least one sterile hollow flow channel for the passage of the flow of blood from the patient 11 therethrough. The input tube 12 and the output tube 16 may each comprise any biologically- or blood-compatible extracorporeal tube or like member. In some embodiments, the input tube 12 and the output tube 16 may be formed of a biologically- or blood-compatible material, such as PVC, polyurethane, ethylene vinyl acetate (EVA), polyacrylonitrile (pAN), silicone, a thermoplastic elastomer (TPE) or a combination thereof, for example.

In some embodiments, the input tube 12 and/or the output tube 16 may include at least a portion that is transparent or translucent such that the flow of blood therethrough is visible to the naked eye. In some embodiments, the input tube 12 and/or the output tube 16 may be flexible to form a flexible flowpath extending between and connecting the patient 11 and the system 10. In one embodiment, the input tube 12 and the output tube 16 are thin, flexible, plastic hoses.

In some embodiments, the input tube 12 is a continuous integral tube, and the output tube 16 is a continuous integral tube that is separate and distinct from the input tube 12 but in fluid communication with the input tube 12. In some other embodiments, the input tube 12 and the output tube 16 are portions of a single continuous integral tube. In some other embodiments, at least one of the input tube 12 and the output tube 16 are formed of a plurality of interconnected tubes (in fluid communication).

The input tube 12 and/or the output tube 16 may be fluidically coupled to a blood vessel (e.g., vein or artery) of the patient for the flow of blood therefrom or thereto, respectively. The input tube 12 and the output tube 16 may be fluidly coupled to the same blood vessel of the patient 11 (e.g., different portions thereof), or fluidly coupled to different blood vessels of the patient 11. In some embodiments, the input tube 12 and/or the output tube 16 may be fluidly coupled to a blood vessel of the patient 11 via a needle or catheter, for example.

The input tube 12 and the output tube 16 may form an internal passageway that allows for the flow of the blood therethrough. For example, the cross-sectional size and shape of the internal passageways formed by the input tube 12 and the output tube 16 may be configured to allow the blood to flow therethrough without clogging or clotting of the blood (which may be related, at least in part, to the pressure and/or flow rate of the blood within the tube via the at least one pump 20). In some such embodiments, the input tube 12 and the output tube 16 may form an internal passageway with a minimum internal cross-sectional area of at least about 1.25 square millimeters (mm²). As a further example, the surface finish and/or material of the internal passageway of the input tube 12 and the output tube 16 may be configured to allow the blood to flow therethrough without clogging or clotting of the blood (which may be related, at least in part, to the pressure and/or flow rate of the blood within the tube via the at least one pump 20). For example, the internal surfaces of the input tube 12 and the output tube 16 that form the internal passageways thereof may be substantially smooth (e.g., comprise a surface roughness of 0.5 micrometer (μm) Ra or less) and/or include an low friction and/or hydrophobic substance (e.g., polytetrafluoroethylene, fluorinated ethylene propylene, manganese oxide polystyrene nano-composites, zinc oxide polystyrene nano-composites, fluorinated silanes and silica nano-coatings).

The extracorporeal blood pump 20 may be configured to draw a flow of blood from the patient 11 via the input tube 12 and to pass a flow of blood from the patient 11 through at least the extracorporeal disinfection device 14 (and any other potential component of the system 10, as described further below) and back into the patient 11 via an output passageway, tube or flow 16, at least in part. The extracorporeal blood pump 20 may comprise any biologically- or blood-compatible extracorporeal pump mechanism effective to form a flow of the infected blood from the patient 11 via the input tube 12, through at least the extracorporeal disinfection device 14, and back to (and into) the patient 11 via the output tube 16. For example, the extracorporeal blood pump 20 may comprise a centrifugal extracorporeal blood pump, a roller extracorporeal blood, a pulsatile tube compression extracorporeal pump, a ventricular extracorporeal pump or another pump type or pump configuration (such as another peristaltic pump configuration). However, it is noted that any blood pump may be utilized.

As shown in FIGS. 1 and 2, the blood pump 20 may be positioned between the patient 11 and the extracorporeal disinfection device 14 along the input tube 12 in the direction of the flow of the blood. In some such embodiments, the blood pump 20 may act on the input tube 12 to form, at least in part, the flow of the blood therethrough. In some alternative embodiments (not shown), the blood pump 20 may be positioned between the extracorporeal disinfection device 14 and the patient 11 along the output tube 16 in the direction of the flow of the blood. In some such embodiments, the blood pump 20 may act on the capillary to form, at least in part, the flow of the blood therethrough. In some other embodiments (not shown), the system 10 may include a plurality of blood pumps 20, which may be positioned before and/or after the extracorporeal disinfection device 14 in the direction of the flow of the blood.

In some embodiments, as shown in FIGS. 1 and 2, the system 10 and method may include a blood pump 20 with a built-in or attached extracorporeal disinfection device 14 with a microbicidal lighting device 24 intended to expose blood flow at sufficient intensity and duration that is effective in reducing or eliminating or slowing the proliferation of a broad range of infectious sources, such as those associated with sepsis, severe sepsis and/or septic shock.

In some embodiments, as shown in FIG. 1, the system 10 and method may include at least one input 30 positioned upstream of the extracorporeal disinfection device 14, such as proximate to the patient 11. The input 30 may introduce at least one substance into the flow of blood flowing through the input tube 12. In one embodiment, the input 30 may comprise a substance that aids or facilities the flow of the blood through the input tube 12, extracorporeal disinfection device 14 and/or the output 16. For example, the input 30 may comprise an anticoagulant pump or supply (e.g., a heparin pump or supply) that decreases the clotting ability of the blood to prevent clots from forming in the input tube 12, the extracorporeal disinfection device 14 and/or the output 16 that restrict the flow of the blood therethrough. As another example, the input 30 may comprise blood thinner pump or supply that increases the amount of time it takes the blood to clot and/or reduces the viscosity of the blood to prevent restriction of the flow of the blood. In some embodiments, the least one input 30 may be positioned downstream of the extracorporeal disinfection device 14, or comprise at least one first input 30 positioned upstream of the extracorporeal disinfection device 14 and at least one second input 30 positioned upstream of the extracorporeal disinfection device 14.

In some embodiments, as shown in FIG. 1, the system 10 and related method may include a separator 31 that separates the blood of the patient 11 into component parts, such as plasma, white blood cells (WBCs) and/or red blood cells (RBCs). One or more selected portions of the blood composition may thereby flow to the extracorporeal disinfection device 14 and be treated thereby, as shown in FIG. 1. Whole blood or separated portions of blood passing through/by the light source may thereby be effectively disinfected by the light's microbicidal action by one or more of the following effects: elimination of pathogenic microorganisms, reduction in the number of pathogenic microorganisms, and reducing the rate of proliferation of pathogenic microorganisms. As also shown in FIG. 1, one or more separated portion of the flow of blood isolated by the separator 31 may be directed downstream of the extracorporeal disinfection device 14 and rejoined with the treated portion of the flow of blood in the output tube 16. In some embodiments, the separator 31 may comprise one or more semipermeable membranes, comprised of cellulose acetate, nitrocellulose, polysulfone, or other membrane material, and containing variable or fixed size pores or openings. In other embodiments, the separator 31 may comprise one or more microfluidic channels. A vacuum pump, microfluidic pump, or other pump mechanism may be used to create a pressure gradient to draw material through the separator.

In some embodiments, as shown in FIG. 1, the system 10 and related method may include at least one pressure monitor 36. The pressure monitor 36 may be positioned upstream of the extracorporeal disinfection device 14 (and downstream or upstream of the at least one pump 20), as shown in FIG. 1, and/or downstream of the extracorporeal disinfection device 14. The pressure monitor 36 may be any pressure monitoring device configured to detect (and potentially display or otherwise indicate) the blood pressure of the patient 11, the pressure of the blood flowing through the input tube 12, the pressure of the blood flowing through the extracorporeal disinfection device 14 and/or the pressure of the blood flowing through the output tube 16. In some embodiments, the system 10 and related method may utilize blood information from the at least one pressure monitor 36 to determine the operation of the pump 20, the input 30 and/or the treatment or infusion material 35 to maintain the blood pressure within a particular range, for example.

As also shown in FIG. 1, in some embodiments the disinfection systems and methods include a filter 38 configured to remove, bind to, chemically bind to, or otherwise filter out at least one substance, component and/or or portion from the flow of blood. In one embodiment, the filter 38 may be built into or attached to the output tubing 16 returning the treated blood to the patient 11 downstream of the extracorporeal disinfection device 14, as shown in FIG. 1. In this way, the filter 38 may remove or otherwise filter out at least one substance from the treated blood, such as one or more portion of the blood that is altered, destroyed, or otherwise effected by the light treatment of the extracorporeal disinfection device 14. For example, the filter 38 may be configured to filter out at least one constituent part or substance of the disinfected blood flowing through the output tube 16 that has become degraded due to the dose of the emitted light from the extracorporeal disinfection device 14.

In some embodiments, the filter 38 may be positioned upstream of the extracorporeal disinfection device 14 to remove or otherwise filter out at least one substance from the untreated blood flowing through the input tube 12. In some embodiments, at least a first filter 38 may positioned upstream of the extracorporeal disinfection device 14 to remove or otherwise filter out at least one substance from the untreated blood flowing through the input tube 12, and at least a second filter 38 may positioned downstream of the extracorporeal disinfection device 14 to remove or otherwise filter out at least one substance from the treated blood flowing through the output tube 16.

In some embodiments, the filter 38 may comprise a filter medium that defines a plurality of passageways therethrough of a particular size or ranges of sizes. The filter may thereby be configured to prevent components or portions of the flow of blood from flowing therethrough that are larger than the size(s) of the passageways to filter out the components or portions from the blood. As another example, the filter 38 may comprise a substance that binds, bonds or otherwise couples to one or more components or portions of the flow of blood flowing therethrough or thereover. The filter may thereby be configured to prevent components or portions of the flow of blood from flowing therethrough or thereover that bind to the substance to filter out the components or portions from the blood. In some embodiments, the filter 38 may comprise aluminosilicates, molecular sieves, activated charcoal, silicalite, zeolite, composite materials comprising a selective molecular absorber (such as above) and a binder agent, such as a polymer, zeolite-polymer composites, nanofiber mesh, semipermeable membrane (e.g., that is selective to molecules based on size), synthetic ion channel membranes (e.g., that is selective to molecules based on size and/or polarity/solubility) or a combination thereof as filter media.

In some embodiments, filter 38 may comprise elements coated in a chemical binder agent. This binder agent may comprise a compound that selectively binds to components of pathogenic or single celled organisms, a compound that selectively bonds to endotoxins, or a compound that binds to other undesirable toxins found within the patient blood. The filter may comprise a binder agent such as polymyxin B (PMX), immobilized onto fibers or other physical structures that present a large surface area to the treatment blood flow, and contained within a cartridge, tube, reservoir, or other physical enclosure that directs the treatment blood flow.

In some embodiments, filter 38 may comprise one or more different filter components connected serially such that each sequential filter component removes a different toxin or detrimental byproduct from the blood. For example, filter 38 may comprise first a PMX binding component, and a secondly a synthetic ion channel membrane component. In other configurations, multiple filter components may be connected in parallel to increase overall flow through the system or to decrease pressure drop across the filter 38.

As shown in FIG. 1, the system 10 may include a manifold or supplemental input 34 positioned downstream of the extracorporeal disinfection device 14. The manifold 34 is configured to input at least one substance into the flow of blood flowing through the output tube 16. For example, if the system 10 includes the separator 31 described above, the manifold 34 may reintroduce the material/portion of the blood that was separated by the separator 31 into the flow of treated blood flowing through the output tube 16, as shown in FIG. 1. In such embodiments, the manifold 34 may be in fluid communication with the separator 31 via a bypass tube or other passageway that carries or contains a flow of the material separated from the flow of pre-treated/infected blood by the separator 31, as shown in FIG. 1.

In some embodiments, the manifold 34 may be in fluid communication with a supply of treatment or infusion material 35 configured to treat the treated/disinfected blood. The manifold 34 may thereby introduce the treatment material 35 into the flow of treated/disinfected blood flowing through the output tube 16. In one embodiment, the dose of light from the extracorporeal disinfection device 14 may degrade at least one constituent part or substance of the pre-treated/infected blood, and the treatment or infusion material 35 may comprise the at least one constituent part or component of the infected blood in a non-degraded state and/or comprises a substance that treats the degraded at least one constituent part or substance of the infected blood.

As also shown in FIG. 1, in some embodiments the disinfection system 10 and related methods include at least one air trap or air remover 33 configured to trap or otherwise remove or eliminate air and from the flow of blood. In one embodiment, the air trap 33 may be built into or attached to the input tubing 12, the extracorporeal disinfection device 14 and/or the output tubing 16. The at least one air trap or air remover 33 is configured to remove air or other gases from the flow of blood flowing through the system 10 such that the gas(es) do not reach the patient 11. The air trap or air remover 33 may be any mechanism or configuration effective is removing or otherwise preventing air or other gases from remaining within the flow of blood and, ultimately, flowing to the patient 11 in the flow of treated blood in the output tube 16. In some embodiments, the system 10 may include at least one air trap or air remover 33 downstream of the extracorporeal disinfection device 14, as shown in FIG. 1.

In some embodiments, the disinfection system 10 may be designed to meet a determined minimum rate of disinfection, dosage, or other treatment criteria set by the operator. The rate of disinfection is determined by a number of factors, including the characteristics of the substrate (blood), the specific pathogen(s) involved, the wavelengths of light utilized, the relative ratios between energy at said wavelengths, the optical transmission of the irradiation chamber and other elements of the system, the geometry of the irradiation chamber, and the rate of flow through the system.

Some of these are ‘external factors’ are not attempted to be controlled directly by the system, but rather compensated for. For example, the optical transmission of the blood is known to vary between patients with different health conditions. For example, iron deficiency anemia can result in increased transmission of wavelengths in the 600-800 nm range, while dehydration can result in decreased transmission of the same range. Drugs can also impact the optical characteristics of patient blood, especially drugs intended for use in photodynamic therapy or that otherwise function as a photosensitizing agent. The physical characteristics of the blood also play an important role as a limiting factor in the geometry of the irradiation chamber and other extracorporeal loop components. TABLE 2 lists sample transmission data for a healthy human male at various wavelengths, expressed as a percentage of 400 nm transmission in the same sample. This data illustrates that 630 nm and 660 nm light are transmitted substantially better than 400 nm through the 0.02 mm and thicker sample depths.

TABLE 2
Transmission relative to 400 nm at various
blood depths in healthy human male blood:

| | Blood depth (mm) | | | | | | | | |
|---|---|---|---|---|---|---|---|---|---|
| | 0 | 0.02 | 0.04 | 0.05 | 0.06 | 0.08 | 0.1 | 0.13 | 0.15 |
| 400 nm | 100% | 100% | 100% | 100% | 100% | 100% | 100% | 100% | 100% |
| 495 nm | 100% | 274% | 549% | 550% | 1515% | 960% | 818% | 534% | 406% |
| 525 nm | 100% | 255% | 484% | 440% | 1142% | 615% | 459% | 334% | 272% |
| 560 nm | 100% | 206% | 439% | 368% | 953% | 434% | 279% | 228% | 197% |
| 630 nm | 100% | 329% | 1074% | 1393% | 4906% | 5130% | 6571% | 7571% | 7214% |
| 660 nm | 100% | 371% | 1125% | 1466% | 5190% | 5519% | 7135% | 8208% | 8141% |

Some patient health conditions can change the viscosity of blood, or the likelihood of clotting.

An embodiment of this technology includes the use of an anticoagulant agent such as heparin (UFH), low molecular weight heparin (LMWH), argatroban, regional citrate (RCA), or other agents to prevent clotting within the system. Unless contraindicated by specific health conditions, UFH is preferred and approved for related uses (dialysis) in the United States by the Food and Drug Administration (FDA) because it offers safe reduction of clotting risk with a short half-life. Use of UFH or other short half-life anticoagulant agents requires continued or periodic administration of the agent during extracorporeal treatment to maintain the anti-clotting effects, whereas some other agents such as LMWH may be administered before treatment, and the effects last throughout treatment without additional agent administered.

Table 3 lists weighting factors for reactive oxygen species (ROS) production at various wavelengths in a sample bacterium, normalized to 400 nm.

TABLE 3
Weighting factors D(λ)

| Wavelength Range | Weighting |
|---|---|
| (nm) | Factor |
| 380-394.99 | 0.8 |
| 395-424.99 | 1.0 |
| 425-459.99 | 0.6 |
| 460-494.99 | 0.02 |
| 495-574.99 | 0.15 |
| 575-659.99 | 0.20 |
| 660-800 | 0.005 |

Table 3 can be used with the equation created herein for ‘Effective Delivered Disinfection’ (EDD)—power disinfection dosage in 405 nm light equivalent ROS production of a multiband disinfection treatment. EDD allows for the comparison of the overall efficacy of a multiband light to a 405 nm light for disinfection, accounting for wavelength specific attenuation within a 3-dimensional volume of blood of arbitrary geometry.

### Generic Form of the Equation for EDD:

$$EDD = \int \int {\int }_{S} {\int }_{{λ}_{1}}^{{λ}_{2}} L ( x , y , z , λ ) D ( λ ) d λ dzdydx$$

Where: L(x,y,z,λ)=energy delivered within a body of fluid S at point (x,y,z) and wavelength λ; and D(λ)=weighting factor.

This equation serves to sum up all light energy within the irradiation chamber, at all depths after any attenuation, and after correction for relative efficacy vs 405 nm. The result is the effective dosage/irradiation level in units that match L( ) either power or energy.

### Applied Form of EDD for Common Irradiation Chamber Geometries:

$$EDD = {\sum }_{s} {\int }_{{λ}_{1}}^{{λ}_{2}} {\int }_{0}^{d} \frac{T ( d , λ ) E ( λ ) D ( λ ) A ( d )}{{A}_{s} ( d )} dt$$

This form of the equation assumes each light source s creates a uniform light distribution, and there are an arbitrary number of sources s. The distance d is from the surface of the irradiation chamber, and all points where d=0 may be nonplanar in some configurations (such as with a round tube profile irradiation chamber).

### Here:

s=disinfecting light source,
λ=wavelength,
λ₁ and λ₂ are the bounds of the spectrum produced by a given disinfecting light source s,
d=depth from surface of blood (0 depth is surface closest to the disinfecting light),
T(d,λ)=transmission in blood at a given depth d and wavelength 2,
E(λ)=disinfecting energy or power produced at a wavelength 2 by a given disinfecting light source s,
D(λ)=weighting factor,
A(d)=cross sectional area exposed to disinfecting light at depth D, and
Aₛ(d)=overall area of uniformly distributed light at depth d (including light not incident on irradiation chamber).

The output from this form of the equation is the effective dosage/irradiation level in units that match L( ) either power or energy.

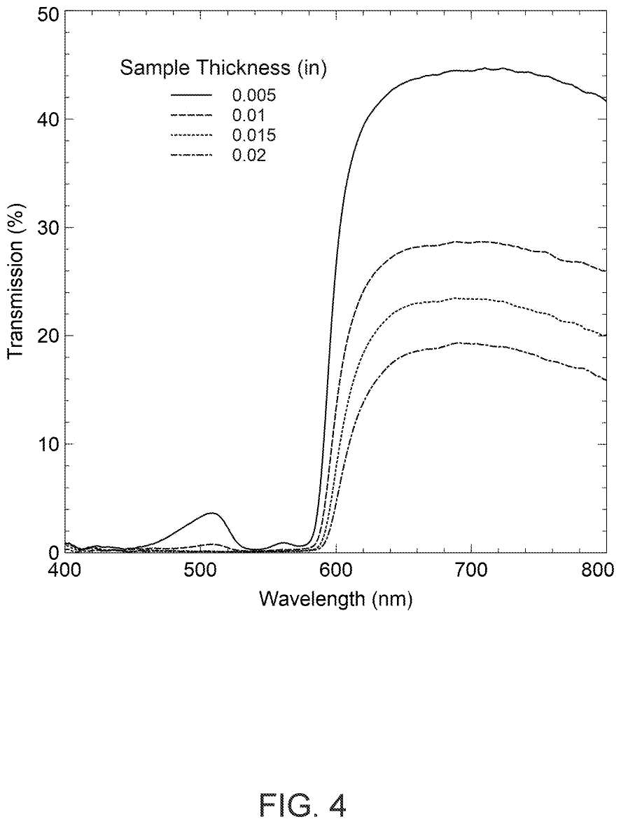

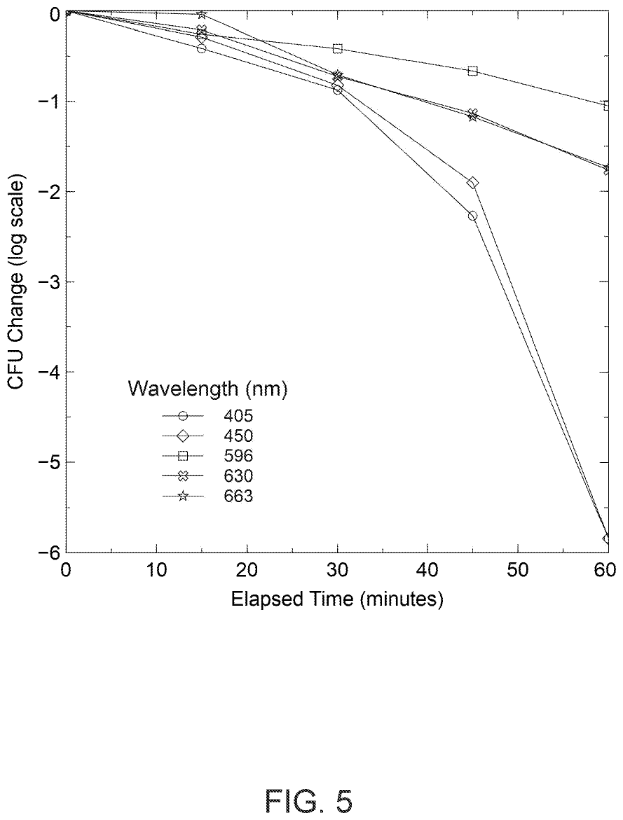

FIG. 4 shows experimental data of transmission percentage by wavelength, for selected transmission depths. At any given wavelength, a mathematical model for transmission as a function of depth d may be determined by statistical techniques, such as nonlinear regression. The range of depths tested should be representative of the maximum depth of the irradiation chamber and is dependent of the fluid being treated (i.e., healthy human blood is expected to have a different mathematical model than anemic human blood or bovine blood). This technique may be used to develop a model for T(d, λ) instead of directly measuring the transmission at every depth.

$${EDD}_{ratio} = \frac{EDD ( multiband )}{EDD ( 400 nm )}$$

The equation for Effective Delivered Disinfection Ratio (EDDᵣₐₜᵢₒ) can be used to compare the relative efficacy of a system versus using 400 nm light alone. This is useful for evaluating the suitability of a given spectrum to a given treatment pathway geometry and allowing for optimization of a multiband disinfection system. For a given overall output power, any EDDᵣₐₜᵢₒ over 1.0 indicates improved performance over using 400 nm light alone. A preferred embodiment of the disclosure utilizes a multiband spectrum where EDDᵣₐₜᵢₒ exceeds 1.25. In certain embodiments, disinfection unit 14 has an effective delivered dosage ratio greater than 1.0. In certain embodiments, disinfection unit 14 has an effective delivered dosage of greater than 4 Watts. EDD represents a minimum effective dosage, regardless of irradiation chamber geometry and wavelengths selected. The EDD ratio >1 means that, with a given geometry, the effective delivered dosage of multiband energy to bacterial cells is greater than the dosage would be using pure 405 nm light in that same geometry chamber.

The extracorporeal disinfection device 14 may be configured in a variety of differing configurations that effectuate a dose of the light emitted by the microbicidal light emitting device 24 to the infected blood flowing through the treatment flowpath 22 that disinfects the blood, such as a dose that is effective in at least one of eliminating pathogenic microorganisms from the infected blood, partially reducing the number of the pathogenic microorganisms in the infected blood, and reducing the rate of proliferation of the pathogenic microorganisms in the infected blood, as described above.

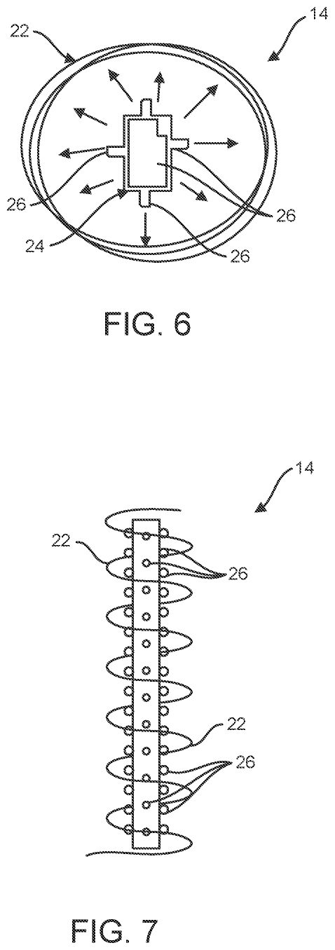

For example, as shown in FIGS. 6 and 7, in some embodiments the extracorporeal disinfection device 14 may comprise a translucent or transparent three-dimensional coil treatment flowpath 22 that extends at least partially about the microbicidal light emitting device 24. In some such embodiments, the coil treatment flowpath 22 may comprise a helical channel or tube that extends at least partially about the microbicidal light emitting device 24, as shown in FIGS. 6 and 7. The treatment flowpath 22 may thereby define a coiled channel or tube that extends along an axial or length direction (as opposed to a planar coil channel) about the microbicidal light emitting device 24.

As also shown in FIGS. 6 and 7, the microbicidal light emitting device 24 may comprise an emitter shaft that is elongated along an axial or length direction with a plurality of light emitting devices 26 provided about an axis of the emitter shaft and/or along the axial length of the emitter shaft. The emitter shaft and/or plurality of light emitting devices 26 are configured to emit light outwardly toward the coiled (e.g., helical) treatment flowpath 22 (and thereby incident on the flow of blood flowing through the treatment flowpath 22.

In some embodiments, the light emitting devices 26 are provided in a helical arrangement as shown in FIG. 7, which may (or may not) mimic, correspond or follow the path of the coiled (e.g., helical) treatment flowpath 22. However, the light emitting devices 26 may be provided in any regular or irregular arrangement on the emitter shaft. The arrangement and number of light emitting devices 26 of the emitter shaft are configured such that the blood flowing through the coiled (e.g., helical) treatment flowpath 22 receive an effective dose of light emitted therefrom, as explained above. In some embodiments, the light emitting devices 26 comprise a plurality LEDs provided on or otherwise coupled to the emitter shaft. In some embodiments, the light emitting devices 26 comprise multiple different wavelength devices, configured such that the overall energy delivered to the treatment flowpath comprises multiple disinfecting wavelengths. In some such embodiments, the emitter shaft includes a thermal management device configured to cool the light emitting devices 26.

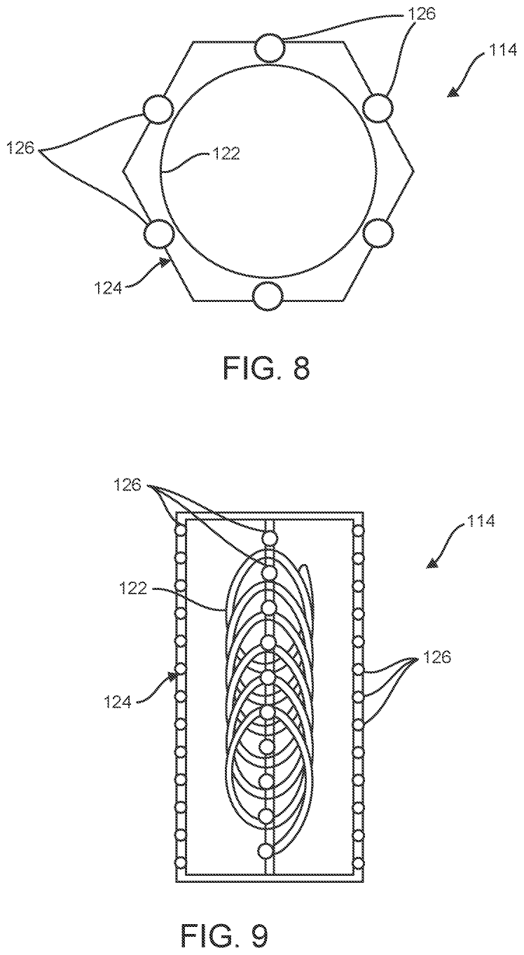

Another illustrative extracorporeal disinfection device 114 is illustrated in FIGS. 8 and 9. As shown in FIGS. 8 and 9, the extracorporeal disinfection device 114 is similar to the extracorporeal disinfection device 14 illustrated in FIGS. 6 and 7, but differs in that the coiled (e.g., helical) treatment flowpath 122 is provided at least partially within the microbicidal light emitting device 124. Stated differently, the microbicidal light emitting device 124 extends at least partially about the coiled (e.g., helical) treatment flowpath 122.

As shown in FIGS. 8 and 9, the microbicidal light emitting device 124 may comprise a hollow or open structure that is elongated along an axial or length direction, and the coiled (e.g., helical) treatment flowpath 122 is positioned at least partially within the internal cavity of the microbicidal light emitting device 124. The plurality of light emitting devices 126 of the microbicidal light emitting device 124 are provided in or face the internal cavity of the microbicidal light emitting device 124, and thereby the coiled (e.g., helical) treatment flowpath 122 (and thereby incident on the flow of blood flowing through the treatment flowpath 122), as shown in FIGS. 8 and 9. The plurality of light emitting devices 126 may thus be provided about and along the exterior of the coiled (e.g., helical) treatment flowpath 122. In some configurations, the light emitting devices may comprise two or more different wavelength devices.

In some embodiments, the open structure may be configured with internal mirrored or reflective surfaces or material that reflect the light emitted from the plurality of light emitting devices 126. The mirrored or reflective surfaces of the open structure may be configured to reflect the light emitted from the plurality of light emitting devices 126 inwardly into the interior of the inner cavity and the coiled (e.g., helical) treatment flowpath 122 therein. In this way, light emitted from the plurality of light emitting devices 126 that passes or is not incident on the coiled treatment flowpath 122 may reflect off one or more of the mirrored or reflective surfaces and, ultimately, act on the treatment flowpath 122 (i.e., become incident on the flow of blood therethrough).

In some embodiments, the light emitting devices 126 are provided in a helical arrangement as shown in FIG. 9, which may (or may not) mimic, correspond or follow the path of the coiled (e.g., helical) treatment flowpath 122. However, the light emitting devices 126 may be provided in any regular or irregular arrangement. The arrangement and number of light emitting devices 126 on/in the open structure are configured such that the blood flowing through the coiled (e.g., helical) treatment flowpath 122 receive an effective dose of light emitted therefrom, as explained above. In some embodiments, the light emitting devices 126 comprise a plurality LEDs provided on or otherwise coupled to the open structure.

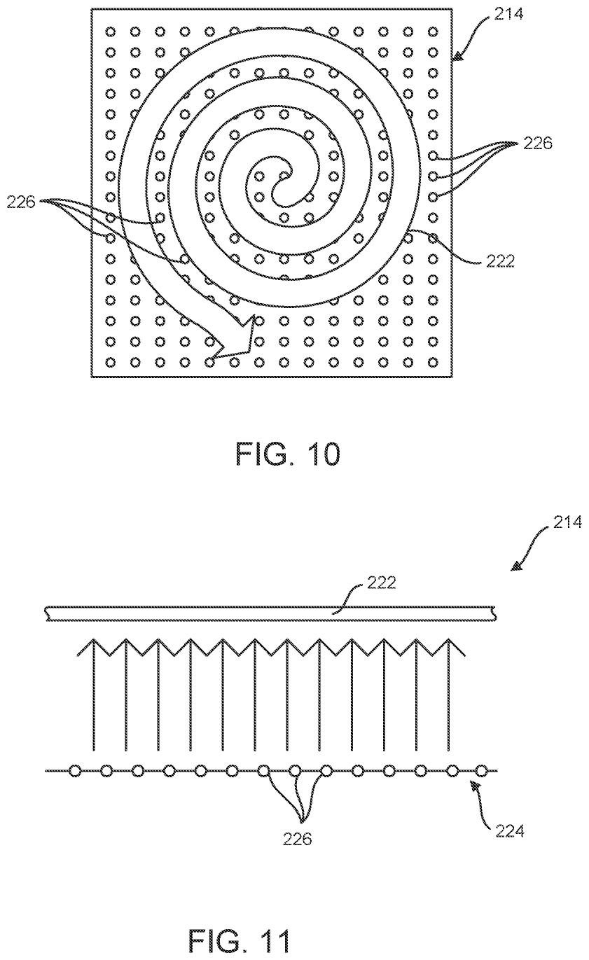

Another illustrative extracorporeal disinfection device 214 is illustrated in FIGS. 10 and 11. As shown in FIGS. 10 and 11, the extracorporeal disinfection device 214 may comprise a translucent or transparent two-dimensional coil treatment flowpath 222 that extends adjacent to the microbicidal light emitting device 224. In some such embodiments, the coil treatment flowpath 22 may comprise a planar coiled channel or tube that extends adjacent to the microbicidal light emitting device 224, as shown in FIGS. 10 and 11. The treatment flowpath 222 may thereby define a flat or planar two-dimensional coiled channel or tube that extends along a width and thickness direction, but is flat or planar along an axial or length direction (as opposed to a three-dimensional or helical coil channel) positioned adjacent the microbicidal light emitting device 224 along the axial direction, as shown in FIGS. 10 and 11.

As also shown in FIGS. 10 and 11, the microbicidal light emitting device 224 may comprise an emitter plate that with a plurality of light emitting devices 226 that is positioned adjacent the two-dimensional coiled treatment flowpath 222 along the axial direction. In some embodiments, the emitter plate may be planar. The emitter plate and/or plurality of light emitting devices 226 are configured to emit light outwardly toward the two-dimensional coiled treatment flowpath 222 (and thereby incident on the flow of blood flowing through the treatment flowpath 222).

In some embodiments, the light emitting devices 226 are provided in a regular pattern on the emitter plate, as shown in FIG. 10. However, the light emitting devices 226 may be provided in any regular or irregular arrangement on the emitter plate. The arrangement and number of light emitting devices 226 of the emitter plate are configured such that the blood flowing through the two-dimensional coiled treatment flowpath 222 receives an effective dose of light emitted therefrom, as explained above. In some embodiments, the light emitting devices 226 comprise a plurality LEDs provided on or otherwise coupled to the emitter plate. In some such embodiments, the emitter plate includes a thermal management device configured to cool the light emitting devices 226 and/or a reflective surface that is configured to reflect light emitted from the light emitting devices 226 to/toward the two-dimensional coiled treatment flowpath 222. In some embodiments, the emitter plate may include a thermal sensor configured to regulate the thermal management device.

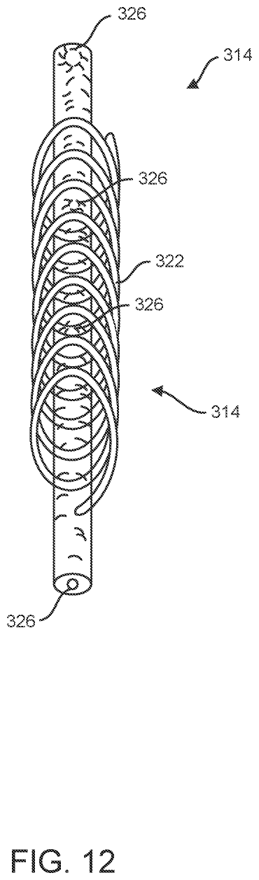

Another illustrative extracorporeal disinfection device 314 is illustrated in FIG. 12. As shown in FIG. 12, the extracorporeal disinfection device 314 is similar to the extracorporeal disinfection device 14 illustrated in FIGS. 7 and 8 but differs in that the light emitting devices 326 are positioned within the emitter shaft and/or are configured to emit light into the emitter shaft. Stated differently, the microbicidal light emitting device 324 comprises an emitter shaft that includes light emitting devices 326 that emit light within or into an interior of the emitter shaft, as shown in FIG. 12. The emitter shaft may comprise a light pipe configured to direct the light from the emitted to the light emitting devices 326 to the coiled (e.g., helical) treatment flowpath 322 extending about the emitter shaft. In some embodiments, at least one light emitting device 326 may be positioned at, proximate to, or within an end portion of the emitter shaft and emits light into and/or through the emitter shaft along the axis of the emitter shaft, as shown in FIG. 12. In some embodiments, at least one light emitting devices 326 may be positioned within the emitter shaft and emit light into and/or through the emitter shaft along the axis of the emitter shaft, as shown in FIG. 12. In such embodiments, the emitter shaft is configured to redirect the light emitted from the at least one light emitting device 326 in an outward radial direction extending away from the longitudinal axis of the emitter shaft, and thereby toward the coiled (e.g., helical) treatment flowpath 322 extending about and along the longitudinal axis of the emitter shaft.

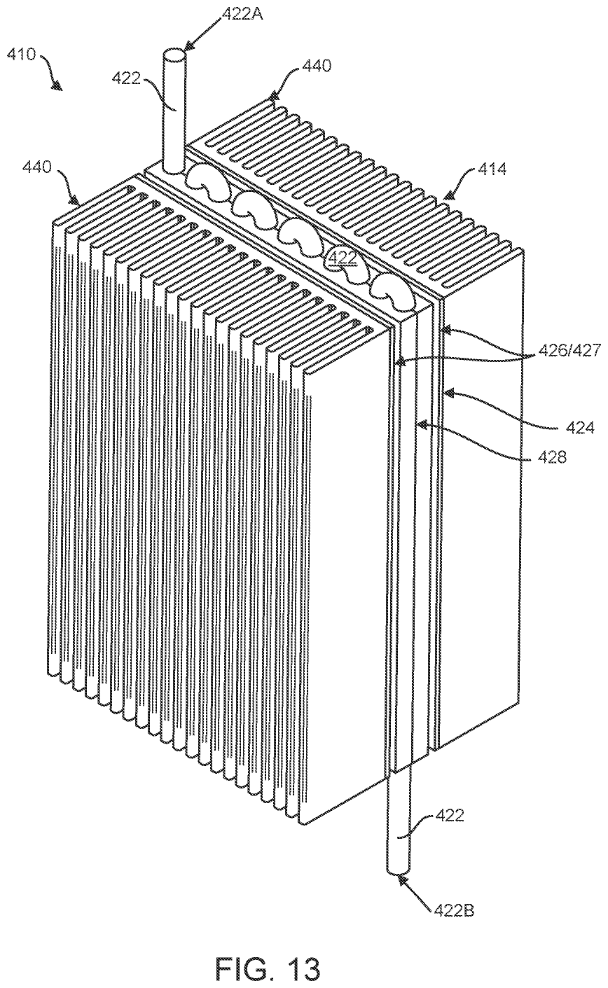

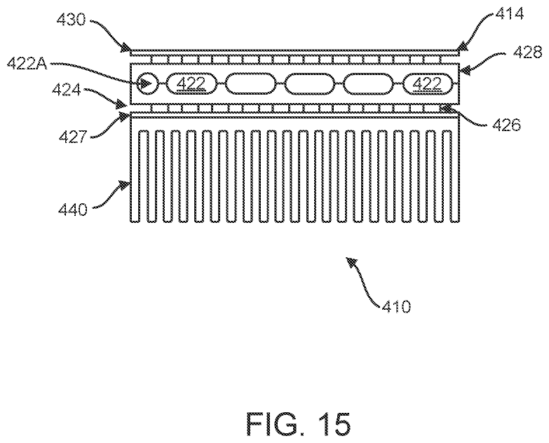

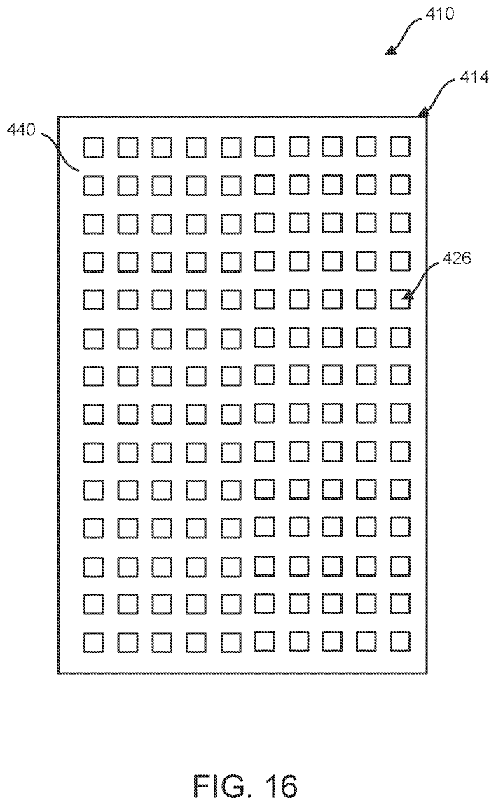

Another embodiment of an extracorporeal blood disinfection system 410 with an extracorporeal disinfection device 414 according to the present disclosure is shown in FIGS. 13-16. As shown in FIGS. 13-16, the extracorporeal disinfection device 414 includes a folded channel treatment pathway 422 (FIG. 15) for blood to pass that is in fluid communication with the input and output tubes (not shown). The folded channel treatment pathway 422 is irradiated from one or both sides via the light emitting device 424, which may utilize LEDs as the light emitting device 426 of the microbicidal light emitting device 424.

As shown in FIGS. 13-16, the microbicidal light emitting device 424 may comprise a plurality or set of light emitting devices 426 (e.g., LEDs) on or associated with a printed circuit board 427 that powers and/or otherwise controls, at least in part, the light emitting devices 426. The light emitting devices 426 and the printed circuit board 427 may thereby be mechanically and electrically coupled. For example, the light emitting devices 426 may be provided on a face of the printed circuit board 427, as shown in FIGS. 13-16. The arrangement of the light emitting devices 426 is configured such that the light energy hitting the channel treatment pathway 422 is substantially uniform, while still achieving a target amount of total energy (i.e., dose), as described above.

In some embodiments, the printed circuit board 427 may be built on, incorporate, or be coupled to a thermally conductive substrate 440 (e.g., a metal or ceramic substrate) to enhance thermal conduction of the circuit board 437 (which may be or comprise fiberglass, for example) and/or the light emitting devices 426, as shown in FIGS. 13-16. The increased thermal conduction and/or convection provided by the substrate 440 may allow for a greater amount of energy to be utilized without overheating the blood in the channel treatment pathway 422. As shown in FIGS. 13-15, in some embodiments the substrate 440) may comprise one or more heatsinks or other thermal management devices that dissipate heat from the printed circuit board 427 and/or the light emitting devices 426, such as from the light emitting devices 426 through the associated circuit board 427. In some embodiments, one or more fans may be used to force air over a substrate 440 to enhance its ability to dissipate heat from the printed circuit board 427 and/or the light emitting devices 426.

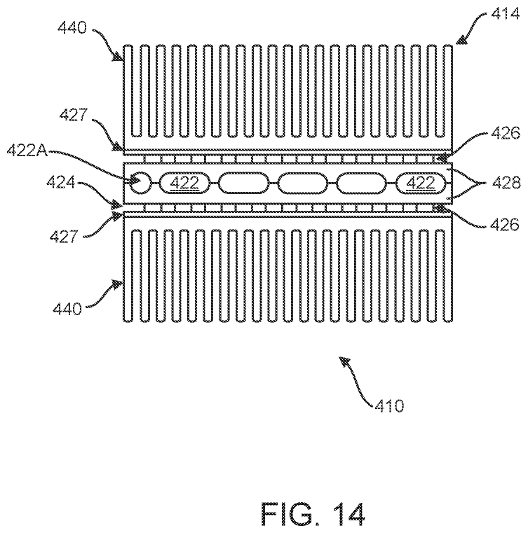

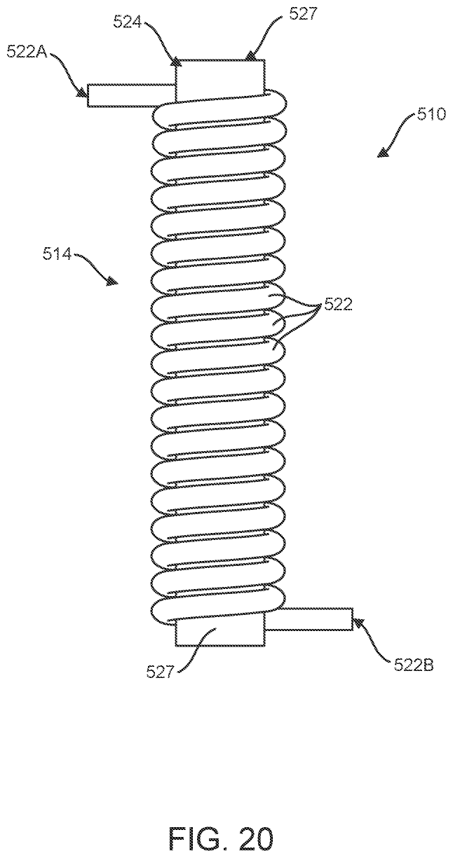

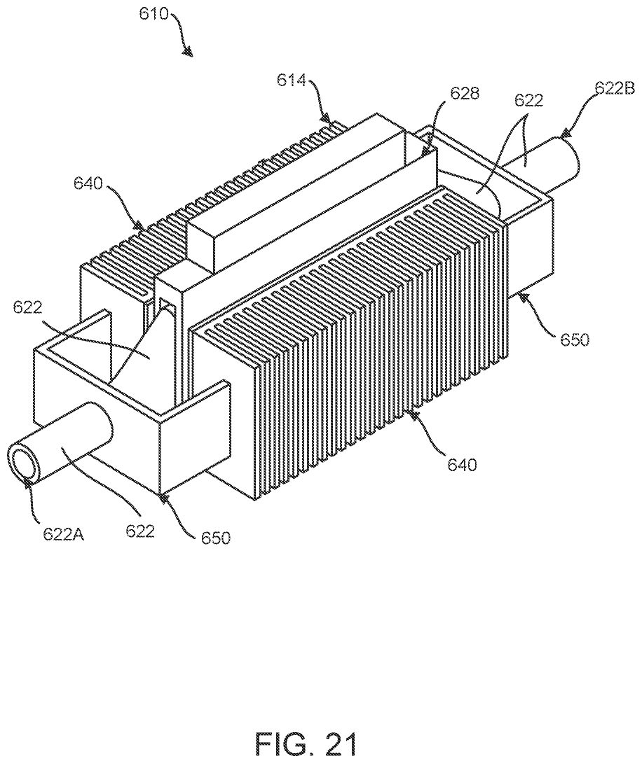

In some embodiments, the extracorporeal disinfection device 414 includes an optical diffuser 428 associated with the microbicidal light emitting device 424, as shown in FIGS. 13-15. The optical diffuser 428 is configured to scatter the light emitted from the at least one light emitting device 426 such that the emitted light incident on the infected blood within the treatment flowpath 422 is of a substantially uniform intensity. In some embodiments, the optical diffuser 428 may comprise one or more polymeric components, and may be configured to scatter light from the LEDs or other irradiation source of the at least one light emitting device 326 to form a substantially uniform intensity on the treatment flowpath 422. The optical diffuser 428 may function through use of aggregate scattering materials within or on the polymer, surface microstructures that scatter the light, molded lens features, for example, or other mechanisms that effectively alter the direction of light emitted from the at least one light emitting device 426. In some embodiments, the treatment flowpath channel 422 may be irradiated from two or more sides via the microbicidal light emitting device 424. For example, the extracorporeal disinfection device 414 may include a pair of microbicidal light emitting devices 424 on two sides (e.g., opposing sides) of the treatment flowpath channel 422, as shown in FIGS. 13 and 14. In some other embodiments, the treatment flowpath channel 422 may be irradiated from only one side via the treatment flowpath channel 422, as shown in FIG. 20. In such embodiments, a reflector 430 may be used on the side void of the microbicidal light emitting device 424/at least one light emitting device 426, as shown in FIG. 20, in order to reflect stray light back into/to/toward the treatment flowpath channel 422 and/or to improve uniformity of irradiation to the flow of blood therethrough.

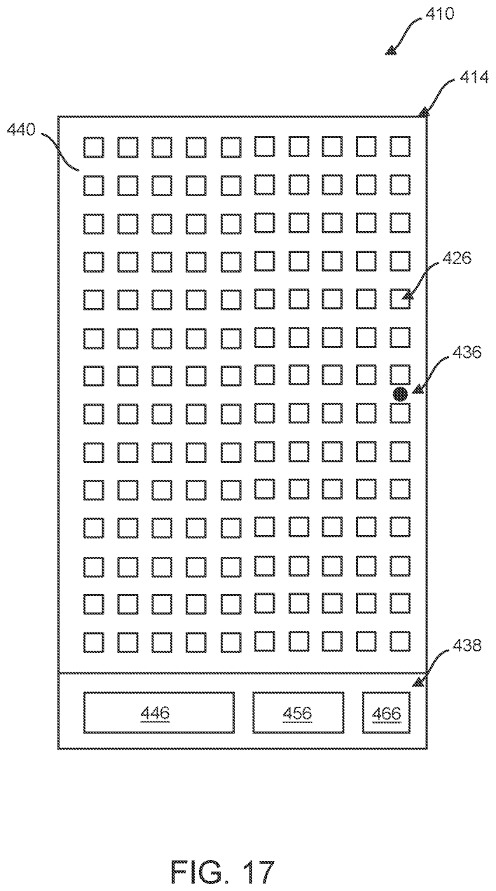

Another embodiment of the extracorporeal disinfection device 414, the light emitting devices 426 may be attached to a substrate 440, illustrated in FIG. 17. A light sensor 436 may be configured to measure transmission of light through the treatment flowpath. This sensor 436 may comprise a photodiode, cadmium sulfide sensor, spectrometer, color sensor, or other sensor that can detect light energy in the disinfecting wavelengths used. This sensor 436 may additionally comprise a filter element, configured to pass one or more disinfecting wavelengths specifically. The sensor 436 may additionally comprise multiple detector elements. These multiple detector elements may be configured with multiple filters, allowing independent measurement of various wavelength ranges. A circuit 438 may be additionally incorporated onto the printed wiring board or connected proximate or located separately but electrically connected to the circuit board 427, optimally positioned offset from the treatment pathway or in a location where the circuit does not obstruct light delivery to the treatment pathway, or prevent the delivery of disinfecting wavelengths to any portion of the treatment pathway. This circuit 438 may comprise a power supply 446. Control circuitry 456 may be used to modulate the output of the power supply 446 to maintain target thermal operating conditions, deliver a preset or operator configured dosage, regulate current to the light emitting devices 426, and monitor treatment delivery. Interface 466 allows an operator to set treatment parameters and monitor treatment. This interface may include a screen, touchscreen, pushbuttons, rotary encoders, or other interface elements. In other configurations, interface 466 may be located remotely from extracorporeal disinfection device 414, and a single interface may be configured to control and monitor multiple sets of light emitting devices and sensors.

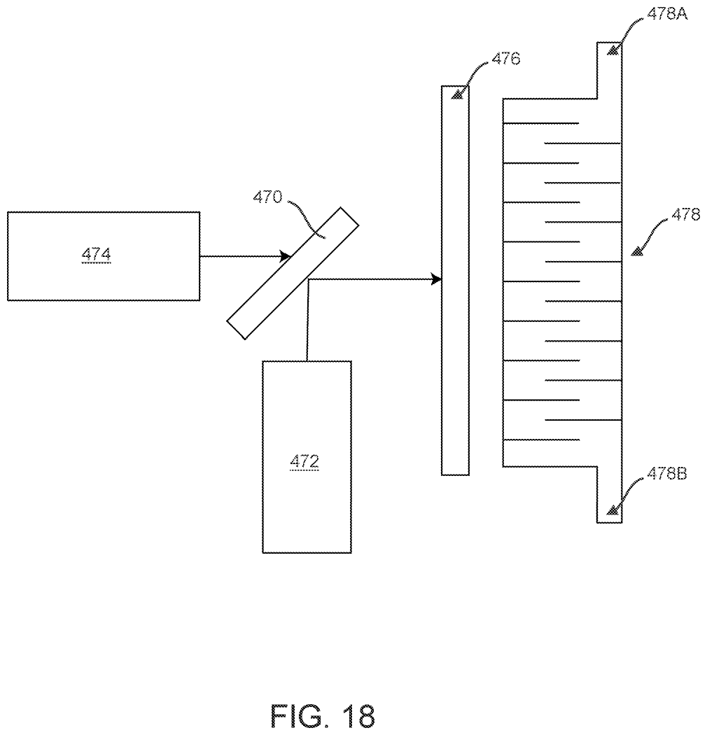

In another embodiment, the light emitting devices may comprise one or more laser sources, illustrated in FIG. 18. In this configuration, a dichroic beam combiner 470 is used to combine the energy from laser source 472 and laser source 474. A light modifier element 476 may be included. Light modifier element 476 may comprise a textured diffuser of PMMA, glass, transparent plastic, clear plastic, acrylic, polycarbonate, or any other light transmissive material, a diffuser with suspended scattering particles, a lens, a diffraction grating, or other optical components or combinations of components, configured to distribute the disinfecting wavelengths across the treatment pathway 478. The treatment pathway 478 is composed of a transparent or translucent material, and comprises a tube, folded channel, flat channel, channel with elements for preventing laminar flow within, or other structure inclusive of at least one inlet 478A and one outlet 478B.

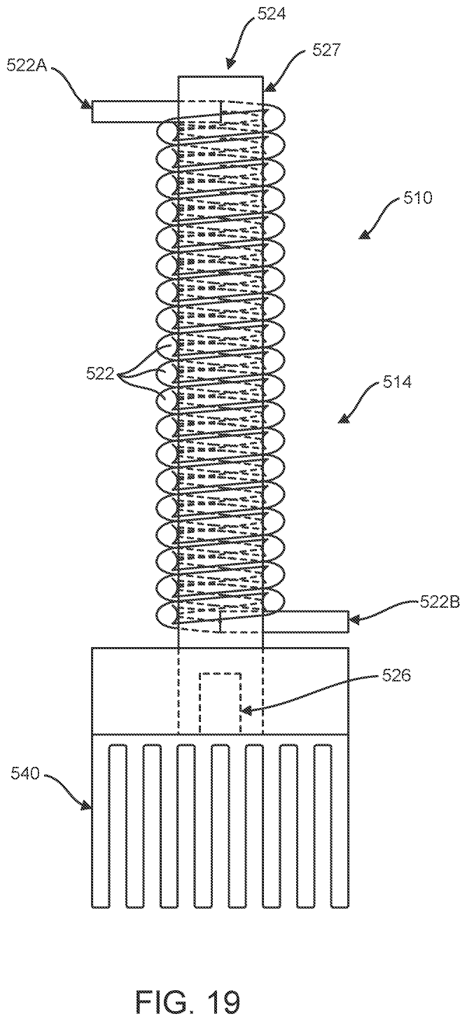

Another embodiment of an extracorporeal blood disinfection system 510 with an extracorporeal disinfection device 514 according to the present disclosure is shown in FIGS. 19 and 20. As shown in FIGS. 19 and 20, in some embodiments, the extracorporeal disinfection device 514 comprises an emitter shaft 527 that is partially surrounded by a helical coiled treatment pathway 522 for the flow of the blood therethrough.

In some embodiments, the emitter shaft 527 may include a central thermal management device. The emitter shaft 527 may be surrounded with outward facing light emitting device 526 as described above with respect to FIGS. 6 and 7. In some other embodiments, the emitter shaft 527 may comprise a light-pipe or other optical element configured to redirect light emitted axially therein/therethrough via at least one light emitting device 526 (e.g., LED) to and through the outer surface of the emitter shaft 527 (i.e., radially) and, ultimately, to the helical coiled treatment pathway 522, as shown in FIG. 19. In such a configuration, at least one light emitting device 526 may be located at one or both ends of the emitter shaft 527. In some embodiments, the light-pipe may comprise a polymeric cylinder or cone, with features such as lenses, ridges, or texture that scatter the light emitted into the emitter shaft 527 outwards toward the helical coiled treatment pathway 522.

The at least one light emitting device 526 may thereby be positioned at an axial end portion of the emitter shaft 527, as shown in FIG. 19. As shown in FIGS. 19 and 20, the helical coiled treatment pathway 522 may be positioned along an opposite end portion and/or medial portion of the emitter shaft 527 than the at least one light emitting device 526 such that the treatment pathway 522 is spaced from the end portion of the emitter shaft 527 including the at least one light emitting device 526 (i.e., axially spaced from the at least one light emitting device 526).

It is noted that the location of the at least one light emitting device 526 at the end of the emitter shaft 527 spaced from the helical coiled treatment pathway 522 limits or attenuates the amount of incidental conduction of thermal energy into the blood flowing through the helical coiled treatment pathway 522 channel from the at least one light emitting device 526. To further limit or prevent the ability or effect of the at least one light emitting device 526 from heating up the blood flowing through the helical coiled treatment pathway 522, the extracorporeal disinfection device 514 may include a heatsink 540 coupled to the at least one light emitting device 526 to remove heat (or exchange heat) therefrom, as shown in FIG. 19.

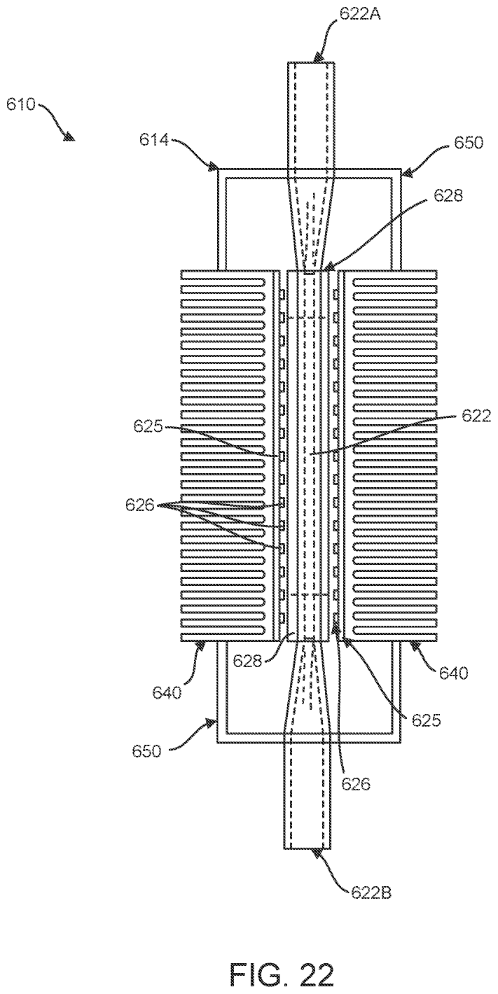

Another embodiment of an extracorporeal blood disinfection system 610 with an extracorporeal disinfection device 614 according to the present disclosure is shown in FIGS. 21 and 22. As shown in FIGS. 21 and 22, the blood disinfection system 610 may include a support bracket 650 that physically supports the extracorporeal disinfection device 614 (and potentially a treatment pathway 622). For example, as shown in FIGS. 21 and 22, the support bracket 650 may physically support and arrange a printed circuit board 625 with a plurality of light emitting devices 626 (e.g., LEDs) and at least one heatsink 640 with respect to the treatment pathway 622, as described above.

As shown in FIGS. 21 and 22, the treatment pathway or channel 622 of the extracorporeal disinfection device 614 includes a flat or narrow portion for the flow of blood therethrough. The narrow portion of the treatment pathway 622 may be formed of a flexible material, such as medium or hard durometer silicone for example. As shown in FIG. 21, the narrow portion of the treatment pathway 622 may include flattened or thin portion that forms a narrow irradiation chamber as compared to an input and/or output portion of the treatment pathway 622. The narrow portion of the treatment pathway 622 may thereby be narrower/thinner in one dimension or axis as compared to the input and/or output portion of the treatment pathway 622, but larger in one or more other dimension or axis such that the cross-sectional area of the treatment pathway is at least as large as the cross-sectional area of the input and/or output portions. In this way, the overall fluidic flow through the treatment pathway 622 may not be affected, or significantly affected, by the narrow portion. The increased surface area of the treatment pathway increases the size of the region where disinfecting wavelengths may be applied, allowing for accommodation of thermal and physical constraints in the light emitting devices. In some embodiments, the treatment pathway 622 (e.g., the narrow/flat portion thereof) may include internal elements to reduce turbulence and to ensure even flow across the wide and/or thin dimensions portions of the narrow portion of the treatment pathway 622. In some embodiments, as shown in FIGS. 21 and 22, the extracorporeal disinfection device 614 may include an optical diffuser 628. The optical diffuser 628 may be positioned proximate to the narrow portion of the treatment pathway 622, as shown in FIGS. 21 and 22. For example, the optical diffuser 628 may be directly coupled to at least a portion of the narrow portion of the treatment pathway 622.

In some implementations of the disclosure, a further improved thermal management system is provided. Maintaining the blood at normal body temperature (approximately 37 degrees Celsius) is critical to patient safety during extracorporeal disinfection. In some configurations, cooling the light emitter using a passive device such as a heatsink is adequate. In higher light output emitters, lower efficiency emitters, or emitters that produce a larger amount of heat, active cooling of the emitter may improve performance (e.g., adding a fan to force air onto the emitter heatsink). Cooling the emitter to a further degree may be necessary if the desired optical flux is substantially higher, for example, if the surface area of the irradiation chamber is decreased to minimize extracorporeal blood volume, or for use in portable systems. This further improved cooling of the emitter can be accomplished using chilled air (cooled below ambient operating temperature), liquid cooling (using a high thermal capacity fluid in a heat exchanger to remove heat from the emitter), chilled liquid cooling (using fluid that is cooled to below ambient operating temperature), thermoelectric cooling (such as a peltier heat pump) or other cooling methods that can dissipate the increased thermal load. Even with improved thermal management of the emitter, the blood may still increase in temperature beyond an acceptable range (e.g., 36.5-37.5° C.). This temperature increase may be due to radiative thermal energy from the emitter that is not removed by the emitter thermal management system, conducted thermal energy from the emitter, and/or thermal energy released as a byproduct of the cells in the blood absorbing the disinfecting and other wavelengths of light from the emitter.

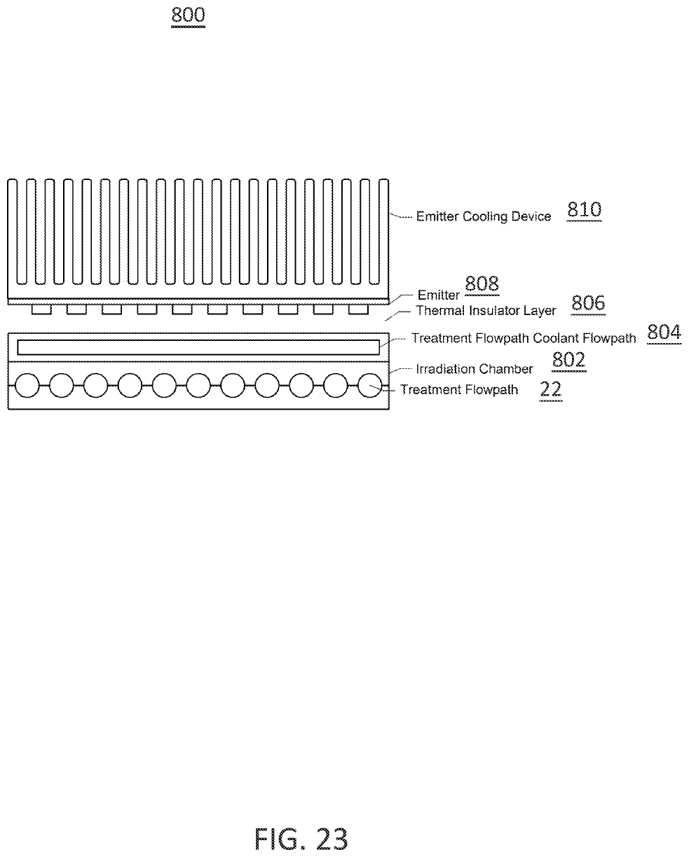

FIG. 23 shows a cross section view of a thermal management system 800 for the emitter and irradiation chamber design that mitigates this blood temperature increase. In this embodiment, an insulating layer 806 is placed between the emitter 808 and the irradiation chamber 802 or treatment flowpath 22. This insulating layer 806 may be any material that is optically transparent to the disinfecting wavelengths and substantially resists conductive heat transfer, including an air gap, vacuum (lack of air or decreased pressure air), inert gas such as nitrogen, or polymeric materials. In some embodiments, an emitter cooling device 810 may be disposed in contact with emitter 808 for cooling of emitter 808. In some embodiments, this insulating layer 806 may also function as an optical diffuser. This insulating layer 806 may also incorporate coatings, layers, or materials that reflect or resist the transmission of infrared thermal energy. In some embodiments the insulating layer 806 may incorporate other optical features, such as micro-lenses, to concentrate or direct the disinfecting wavelengths onto the treatment flowpath.

FIG. 23 also details a treatment flowpath coolant flowpath 804 that is thermally coupled with the treatment flowpath 22 and thermally insulated from the emitter 808. The treatment flowpath coolant flowpath 804 consists of an optically transparent material with one or more channels through which coolant material (e.g., water, antifreeze solution, air, inert gas, refrigerant gas, etc.) can passively or actively be introduced. In some embodiments, two or more treatment flowpath coolant flowpaths 804 may be utilized to minimize temperature gradients across the treatment flowpath. In embodiments with two or more emitters 808, the area on the treatment flowpath exposed by each emitter may have a separate coolant flowpath. Separate coolant flowpaths may be combined at the treatment flowpath or outside the treatment flowpath 22, enabling the use of a single heat exchanger, chiller, or other thermal management system to regulate the temperature.

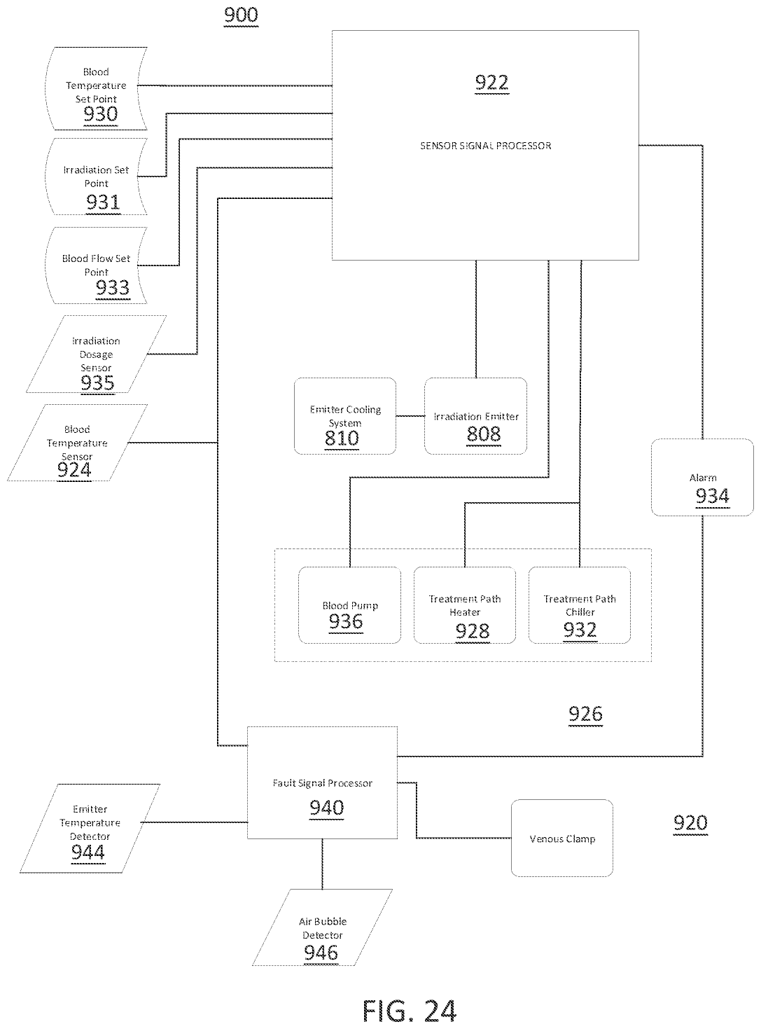

FIG. 24 depicts some elements of the control system 900 in the disclosure with a comprehensive thermal management device 910 and fault detection system 920. Some aspects of the system have been excluded from this diagram, which is intended to illustrate only the thermal control system aspects.

The blood temperature set point is the target temperature or temperature range for the blood to remain in during treatment. This set point may be input by the device operator, preset at the factory, or determined based on device operating mode. The irradiation set point is a maximum allowable temperature for the emitter and is typically preset (not operator adjustable). The maximum emitter temperature may be limited by the technology used for the emitter, insulation characteristics between the emitter and treatment flowpath, and design of the thermal management system. In some implementations, it may be preferable to utilize a higher irradiation set point temperature, to enable a smaller form factor (e.g., for a field portable extracorporeal disinfection unit) with a likely effect of reducing the operating lifespan of the emitter.

The sensor signal processor 922 is a microcontroller, logic circuit, computer, FPGA, or other device that serves to read input signals from the blood temperature sensor 930, emitter temperature detector 944, an irradiation set point 931, a blood flow set point 933, and an irradiation dosage sensor 935, and other sensors and dynamically adjust various outputs in a closed loop control scheme. In this example, the blood temperature sensor 924 and treatment path temperature regulation 926 are accomplished with a closed loop control scheme, and the emitter is cooled with an open loop scheme, with fault detection. In this system, the sensor signal processor 922 continuously or repeatedly compares the current blood temperature sensor 928 value to the blood temperature setpoint value and enables the treatment path heater 928 or treatment path chiller 932 in order to maintain the target set point temperature. If the blood temperature sensor returns a value outside the acceptable range, the device may be configured to activate an alarm 934 and optionally halt operation. The sensor signal processor 922 may alter the speed of the blood pump 936 to mitigate a minor fault (e.g., temporarily increase blood pump 936 speed in a minor blood temperature sensor over temperature event) in addition to activating the appropriate treatment path thermal management elements. In the event of a serious fault (temperature reading is significantly outside of safe operating range and continued use could cause immediate patient harm), a fault signal processor 940 may be used to immediately cease blood flow by activating a venous clamp 942 or other element to rapidly halt operation. Typically, detection of a serious fault would activate an alarm 934 and require intervention by the device operator to continue device operation. Other faults that may activate the fault signal processor include but are not limited to emitter temperature detector 944 out of allowable range (i.e. emitter is overheating and continued operation may damage the emitter), detection of an air bubble by air bubble detector 946 in the treatment flowpath 22, or activation of other sensors that are designed for detecting abnormal conditions that may be detrimental to the patient 10. In some embodiments, the fault signal processor 940 may be a part of the same processor as the sensor signal processor 922.

Not all elements are required in a commercial implementation of the disclosure—some elements may be replaced by characterization of a design. For example, an emitter cooling system may consist of a passive heatsink 1010 (FIG. 23) that requires no control signal from the sensor signal processor. In this example, the system would need to be designed or characterized to ensure that the emitter would remain within allowable operating temperature without feedback from the sensor signal processor across all possible settings and uses of the disclosure.

Terminology used herein is for the purpose of describing particular examples only and is not intended to be limiting. As used herein, the singular forms “a,” “an,” and “the” are intended to include the plural forms as well, unless the context clearly indicates otherwise. Furthermore, references to “one example” are not intended to be interpreted as excluding the existence of additional examples that also incorporate the recited features. Moreover, unless explicitly stated to the contrary, the terms “comprising” (and any form of “comprise,” such as “comprises” and “comprising”), “have” (and any form of “have,” such as “has” and “having”), “include” (and any form of “include,” such as “includes” and “including”), and “contain” (and any form of “contain,” such as “contains” and “containing”) are used as open-ended linking verbs. As a result, any examples that “comprises,” “has,” “includes” or “contains” one or more step or element possesses such one or more step or element, but is not limited to possessing only such one or more step or element. As used herein, the terms “may” and “may be” indicate a possibility of an occurrence within a set of circumstances: a possession of a specified property, characteristic or function; and/or qualify another verb by expressing one or more of an ability, capability, or possibility associated with the qualified verb. Accordingly, usage of “may” and “may be” indicates that a modified term is apparently appropriate, capable, or suitable for an indicated capacity, function, or usage, while taking into account that in some circumstances the modified term may sometimes not be appropriate, capable, or suitable. For example, in some circumstances, an event or capacity can be expected, while in other circumstances the event or capacity cannot occur—this distinction is captured by the terms “may” and “may be.” As used herein, the terms “comprising.”” has.” “including.” “containing,” and other grammatical variants thereof encompass the terms “consisting of” and “consisting essentially of.”

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed compositions or methods.

All publications cited in this specification are herein incorporated by reference as if each individual publication were specifically and individually indicated to be incorporated by reference herein as though fully set forth.

Subject matter incorporated by reference is not considered to be an alternative to any claim limitations, unless otherwise explicitly indicated.

Where one or more ranges are referred to throughout this specification, each range is intended to be a shorthand format for presenting information, where the range is understood to encompass each discrete point within the range as if the same were fully set forth herein.

While several aspects and embodiments of the present disclosure have been described and depicted herein, alternative aspects and embodiments may be affected by those skilled in the art to accomplish the same objectives. Accordingly, this disclosure and the appended claims are intended to cover all such further and alternative aspects and embodiments as fall within the true spirit and scope of the disclosure.

1. An extracorporeal blood disinfection system (10), characterized in that the extracorporeal blood disinfection system comprises:
an input tube (12) forming a flowpath (22) for a flow of infected blood from a mammalian patient (11);
a disinfection unit (14) including a microbicidal light emitting device (24) configured to emit a plurality of light emissions for multiband disinfection treatment, each light emission having a wavelength in a range between about 380 nm and about 800 nm;
a treatment flowpath (22) in communication with the input tube (12) that is substantially transparent to the emitted light of the microbicidal light emitting device (24) for receiving at least a portion of the flow of the infected blood therethrough, wherein the microbicidal light emitting device (24) effectuates a dose of the plurality of light emissions to the infected blood flowing through the treatment flowpath (22) to disinfect the blood; and
an output tube (16) in fluidly and physically connected wherein material can flow within the treatment flowpath forming a flowpath for the flow of the disinfected blood from the disinfection unit back to the patient, wherein an Effective Delivered Disinfection (EDD) power disinfection dosage in 405 nm light equivalent reactive oxygen species (ROS) production of a multiband disinfection treatment accounting for wavelength specific attenuation within a 3-dimensional volume of blood of arbitrary geometry is defined by:
$$EDD = \int {\int }_{S} \int {\int }_{{λ}_{1}}^{{λ}_{2}} L ( x , y , z , λ ) D ( λ ) d λ dzdydx$$
where:
λ=wavelength,
L(x,y,z,λ)=energy delivered within a body of fluid S at point (x,y,z) and wavelength λ; and
D(λ)=weighting factor.

2. The system according to claim 1, further comprising a pump (20) configured to at least one of:
provide the flow of infected blood from the patient (11) through the input tube (12);
pass the blood from the input tube (12) through the treatment flowpath (22); and
provide the flow of disinfected blood to the patient (11) through the output tube (16).

3. The system according to claim 1, wherein the disinfection unit further includes a thermal management device (800, 910) associated with the microbicidal light emitting device (24) and configured to maintain the blood within the treatment flowpath (22) below a blood temperature set point.

4. The system according to claim 3, wherein the thermal management device (800, 910) includes a treatment flowpath coolant flowpath (804) thermally coupled with the treatment flowpath, the treatment flowpath coolant flowpath (804) including one or more channels configured to allow a coolant to flow therethrough.

5. The system according to claim 4, wherein the treatment flowpath coolant flowpath (804) is optically transparent, thermally coupled with the treatment flowpath (22), and thermally isolated from the microbicidal light emitting device (24).

6. The system according to claim 4, further including a blood temperature sensor (924) configured to measure a blood temperature of the blood, wherein the thermal management device (800, 910) controls a temperature of the coolant based on the blood temperature.

7. The system according to claim 4, further including a heat exchanger (540, 810) configured to control the temperature of the coolant based on the blood temperature.

8. The system according to claim 3, wherein the thermal management device (800, 910) controls a blood flow rate, the blood flowrate being generated by a pump (20) based on the blood temperature.

9. The system according to claim 1, further including a fault detection system (940) configured generate an alarm (934) in response to at least one of:
a blood temperature exceeding a blood temperature set point;
a temperature of the microbicidal light emitting device (24) being outside of an allowable range; and
detection of an air bubble in the treatment flowpath (22).

10. The system according to claim 1, wherein the disinfection unit (14) further includes a thermal management device (800, 910) associated with the microbicidal light emitting device (24) configured to dissipate heat from the microbicidal light emitting device (24) to maintain a temperature of at least a portion thereof below a predefined temperature such that the microbicidal light emitting device (24) is prevented from heating the blood within the treatment flowpath (22) above a blood temperature set point.

11. The system according to claim 1, further including an insulating layer (806) between the microbicidal light emitting device (24) and the treatment flowpath (22).

12. The system according to claim 11, wherein the insulating layer (806) is optically transparent and resistant to conductive heat transfer.

13. The system according to claim 11, wherein the insulating layer (806) includes at least one of: air, a vacuum, a gas, aerogels, a material configured to trap air pockets, glass wool, and a polymeric material.

14. The system according to claim 11, wherein the insulating layer (806) includes an optical diffuser.

15. The system according to claim 11, wherein the insulating layer (806) includes a surface, the surface configured to provide at least one of reflection or resistance of transmission of infrared thermal energy.

16. The system according to claim 11, wherein the insulating layer (806) includes at least one optical feature to direct the plurality of light emissions.

17. The system according to claim 1, wherein the dose of light is effective in at least one of:
eliminating pathogenic microorganisms from the infected blood;
partially reducing a number of the pathogenic microorganisms in the infected blood; and
reducing a rate of proliferation of the pathogenic microorganisms in the infected blood.

18. The system according to claim 17, wherein the pathogenic microorganisms include microorganisms associated with at least one of sepsis, severe sepsis, and septic shock.

19. The system according to claim 17, wherein the pathogenic microorganisms include at least one of bacteria, fungi, yeast, and a combination thereof.

20. The system according to claim 17, wherein the pathogenic microorganisms include at least one of gram positive bacteria, gram negative bacteria, bacterial endospores, yeast, filamentous fungi, and a combination thereof.

21. The system according to claim 17, wherein the pathogenic microorganisms include at least one of Staphylococcus aureus, Clostridium perfringens, Clostridium difficile, Enterococcus faecalis, Staphylococcus epidermidis, Staphylococcus hyicus, Streptococcus pyogenes, Listeria monocytogenes, Bacillus cereus, Mycobacterium terrae, Acinetobacter baumannii, Pseudomonas aeruginosa, Klebsiella pneumoniae, Proteus vulgaris, Escherichia coli, Salmonella enteritidis, Shigella sonnei, Serratia spp, Bacillus cereus, Clostridium difficile, Aspergillus niger, Candida albicans, Saccharomyces cerevisiae, and a combination thereof.

22. The system according to claim 1, wherein the plurality of light emissions includes light at about 405 nm and light within at least two ranges between about 380 nm and about 420 nm; between about 400 nm and about 415 nm; between about 500 nm and about 700 nm; between about 500 nm and about 520 nm; between about 530 nm and about 555 nm; between about 565 nm and about 590 nm; and between about 615 nm and about 645 nm.

23. The system according to claim 1, wherein the mammalian patient (11) is a human patient.

24. The system according to claim 1, wherein the disinfection unit (14) has an effective delivered dosage ratio greater than about 1.0 as determined by the EDD for multiband irradiation divided by the EDD for irradiation by radiation with a wavelength of 405 nm.

25. The system according to claim 1, wherein the disinfection unit (14) has an effective delivered dosage of greater than about 4 Watts.
